(19) United States
(12) Patent Application Publication
(10) Pub. No.: US 2011/0118197 A1
(43) Pub. Date: May 19, 2011

(54) PREVENTION OF HEPATIC ISCHEMIC REPERFUSION INJURY BY ADMINISTRATION OF SULFATIDES

(76) Inventor: Vipin Kumar Chaturvedi, Rancho Santa Fe, CA (US)

(21) Appl. No.: 12/938,315

(22) Filed: Nov. 2, 2010

Related U.S. Application Data
(63) Continuation-in-part of application No. 11/529,793, filed on Sep. 28, 2006.
(60) Provisional application No. 60/722,184, filed on Sep. 29, 2005.

(51) Int. Cl. A61K 31/70; A61P 9/10; A61P 1/16

(52) U.S. Cl. 514/25

(57) ABSTRACT

Hepatic ischemic reperfusion injury is a major complication of liver transplantation, resectional hepatic surgeries, trauma surgery and shock. Disclosed herein are methods for the prevention and treatment of ischemia and reperfusion injury with the administration of sulfatides. Also disclosed herein are methods of preventing and treating hepatic reperfusion injury by administering an amount of a sulfatide to the body of a patient effective to reduce or prevent the symptoms of the injury.

### CROSS-REFERENCE TO RELATED APPLICATIONS

This application is a continuation-in-part of and claims priority to U.S. patent application Ser. No. 11/529,793, filed Sep. 28, 2006, which claims priority under 35 U.S.C. §119(e) to U.S. Provisional Patent Application No. 60/722,184, filed Sep. 29, 2005, both of which are hereby incorporated by reference in their entirety.

### BACKGROUND OF THE INVENTION

1. Field of the Invention

The present embodiments relate to methods for the prevention and treatment of reperfusion injury. More specifically the present embodiments relate to the prevention and treatment of hepatic ischemic reperfusion injury. Some embodiments relate to use of sulfatides in the prevention and treatment of reperfusion injury.

2. Description of the Related Art

Natural killer T (NKT) cells are a type of CD1d-restricted T cell that display properties of both natural killer cells and T cells. NKT cells express both a T cell receptor (TCR) and NK markers (such as NK1.1) and secrete cytokines rapidly upon stimulation. Most NKT cells recognize lipid antigens presented in the context of the monomorphic MI-IC class I-like molecule CD1d. CD1d-restricted NKT cells are categorized into type I and type II NKT cells. Type I or invariant NKT (iNKT) cells express a semi-invariant TCR encoded by the Vα14-Jα18 gene segments in mice, are strongly reactive with the marine sponge-derived glycolipid α-galactosyl ceramide (αGalCer), and are therefore identified by αGalCer/CD1d-tetramers in flow cytometry. They also recognize bacterial-derived lipids and a self-glycolipid, isoglobotrihexosyl ceramide (iGb3). Thus, iNKT cells display dual reactivity to microbial and self-derived ligands. Type II NKT cells are less well studied. A subset of type II NKT cells is reactive to the self-glycolipid 3-sulfated 13-galactosyl ceramide, an example of a sulfatide. These sulfatide-reactive type II NKT cells have been identified using sulfatide/CD1 d-tetramers in flow cytometry.

NKT cells have been implicated in several aspects of immunity as deficiencies in NKT cells have been linked to the development of cancers and autoimmune diseases like diabetes and atherosclerosis.

### Autoimmune Diseases

Autoimmune diseases affect millions of people worldwide and can have devastating effects on lifespan and quality of life. Despite advances in medical science, many autoimmune diseases have evaded treatment because the mechanisms of disease are complex and poorly understood. Also, unlike most diseases where treatment involves working with the body's immune system to combat a foreign invader, in autoimmune diseases, the immune system itself is exacerbating the problem. This makes any treatment much more difficult because it must address the immune response directly to combat the problem.

In multiple sclerosis, for example, the immune system pathologically recognizes some self-antigens from myelin membranes as foreign and initiates an immune response against them. This results in demyelination, the destructive removal of myelin which is an insulating and protective fatty protein that sheaths nerve cells (neurons). The demyelination in multiple sclerosis is mediated by a T-cell guided immune response that is either initiated from antigen-presenting events in the CNS or induced following the peripheral activation by a systemic molecular mimicry response.

Experimental autoimmune encephalomyelitis (EAE) is a prototypic T-cell mediated autoimmune disease, characterized by inflammation and demyelination in the central nervous system accompanied by paralysis following immunization with myelin antigens, for example, myelin basic protein (MBP), myelin oligodendrocyte glycoprotein (MOG) or proteolipid protein (PLP). EAE shares many pathological and immune dysfunctions with human MS and is a widely accepted model for studying human MS.

Glycolipids can be recognized by T cells in the context of class I WIC-like cell surface proteins known as CD1. Myelin is a rich source of glycolipids, and myelin is the target of an autoimmune process during EAE in which the influence of myelin-derived lipids and their presentation to T cells in the CNS can be easily studied. In order to derive effective treatments for multiple sclerosis, further characterization of glycolipid-reactive T cells is needed. Sulfatide is one of the major glycolipids in myelin and has been shown to bind to CD1d. (Jahng, et al. J. Exp. Med. Vol. 199 Num. 7:947-957, 2004.)

### AIDS

In AIDS, T-cells are systematically depleted by the HIV virus. Like with many autoimmune diseases, the immune system itself tends to advance the disease because the virus is spread through immune cells. Human immunodeficiency virus (HIV) infects CD4+ cells in conjunction with a cellular coreceptor, CXCR4, or CCR5/CCR3. HIV infection of human cells results in loss of CD4+ T lymphocytes as the virus undergoes rapid replication generating mutations in its envelope region of the viral genome. These also include drug resistant mutants as the infected individuals are treated with antiretroviral drugs including zidovudine (AZT), nucleoside reverse transcriptase inhibitor (NRTI), or a non-nucleoside reverse transcriptase inhibitor (NNRTI), and protease inhibitor. Further, there is an exhaustion of the cytotoxic T lymphocytes and the eventual failure of the immune system, both cell mediated and humoral responses, of the infected individual to fight the infection arising from the generation of multiple HIV strains in vivo. The immune system is also exacerbated due to opportunistic infections of the infected individuals that are immune compromised.

The severe combined immunodeficiency mouse transplanted with human fetal thymus and liver tissues (SCID-hu Thy/Liv) is a small animal model that mimics HIV infection in humans both in terms of loss of CD4+ T lymphocytes and high viral replication. The system also enables testing in the laboratory of various drugs to combat HIV infection in vivo in a convenient model system in the absence of confounding factors found in humans. Thus, this system is a useful model for preclinical testing of anti-HIV drugs in vivo prior to undertaking clinical trials in infected humans.

Cytopenia, particularly thrombocytopenia are a major risk factor in HIV infection, heart disease, and cancer. Hematopoietic abnormalities can cause or lead to multiple cytopenia in HIV infected individuals with thrombocytopenia emerging as a major risk factor for morbidity and mortality and even more so in patients also suffering from heart conditions.

### Liver Diseases

Concanavalin A (Con A)-induced hepatitis in the mouse is a well-characterized model of T cell-mediated liver diseases. This model has been extensively used as an excellent model mimicking human T cell-mediated liver diseases, such as autoimmune hepatitis ((Tiegs et al., 1992, JCI, Mizuhara H., JEM, 1994, Toyabe S, JI, 1997). A single injection of Con A is sufficient for the T cell-mediated liver injury in mice (Tiegs et al., 1992, JCI, Mizuhara H., JEM, 1994, Toyabe S, JI, 1997). Serum enzymes and histological evidence of Con A induced hepatitis is observed following 8-24 hours, as shown by elevated serum levels of alanine aminotransferase (ALT) and aspartate amino transferase (AST) and the occurrence of histological evidence of hepatic lesions characterized by a massive granulocytes accumulation, CD4⁺ T cell infiltration and an influx of a relatively small number of CD8⁺ T cells and hepatocyte necrosis/apoptosis (Tiegs et al., 1992, JCI, Mizuhara H., JEM, 1994, Schumann J., 2000, Am. J. Pathol., Chen et al., 2001). Recently, several investigators have implicated hepatic NKT cells in the development of Con A-induced hepatitis. Both Jα18 and CD1d-deficient mice that lack NKT cells are resistant to Con A-induced hepatic injury (Kaneko et al., 2000; Takeda et al., 2000), indicating that classical CD1d-restricted NKT cells that express the iNKT cell receptor are critically involved in the process of Con A induced hepatic injury.

### Reperfusion Injury

Ischemia is a restriction in the blood supply to tissue. When tissue is deprived of blood supply for a period of time the tissue may be damaged both by the initial absence of oxygen and nutrients and by the return of the blood supply after the period of ischemia. The restoration of blood flow after a period of ischemia can be more damaging than the ischemia itself. The damage caused by the returning blood supply is termed reperfusion injury. Reperfusion injuries are caused, in part, by the inflammatory response of damaged tissues. The restored blood flow brings with it neutrophils that become activated and accumulate at the site of injury. In response to the tissue damage, the activated neutrophils release inflammatory factors and free radicals, which result in further tissue damage.

Liver injury following ischemia and reperfusion is a major complication of liver transplantation, hepatic resections, trauma surgery, and shock. Ischemia and reperfusion injury is mediated by a biphasic inflammatory response. The initial phase (following 1-6 hrs of reperfusion) involves Kupffer cell activation, release of reactive oxygen species, CD4⁺ cell recruitment, and secretion of pro-inflammatory cytokines such as TNF-α and IFN-γ. In the later phase (following 6-48 hrs of reperfusion), accumulated neutrophil granulocytes release oxidants and proteases directly injuring hepatocytes and leading to hepatic necrosis. This results in elevated liver enzymes in serum, in the histological picture of hepatic necrosis, and can lead to organ dysfunction.

Hepatic ischemic reperfusion injury develops rapidly, which is not consistent with the timeframe required for conventional T cell responses. Due to the early recruitment of CD4⁺ T cells, consisting of mostly Natural Killer T (NKT) cells, a role for CD1d-restricted NKT cells has recently been suggested. NKT cells express both a T cell receptor and NK markers (such as NK1.1), are mainly CD4⁺ in the liver, and secrete cytokines rapidly upon stimulation.

### SUMMARY OF THE INVENTION

One embodiment relates to a method of treating a patient with symptoms of an autoimmune disease including administering an amount of a sulfatide effective to reduce said symptoms, wherein the autoimmune disease is not multiple sclerosis.

In one aspect of the embodiment, the sulfatide can have following chemical structure:

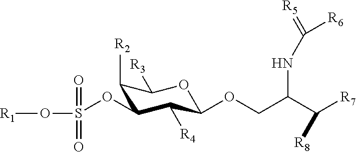

wherein R₁ is selected from the group consisting of a bond, a hydrogen, a C₁ to C₃₀ alkyl, C₁ to C₃₀ substituted alkyl, a C₁ to C₃₀ alkenyl, a C₁ to C₃₀ substituted alkenyl and a C₅ to C₁₂ sugar; R₂ is selected from the group consisting of a hydrogen, a hydroxy group, a methoxy group, and an alkoxy group; R₃ is selected from the group consisting of a hydrogen, a hydroxy group, a methoxy group, an ethoxy group, and an alkoxy group; R₄ is selected from the group consisting of a hydrogen, a hydroxy group and an alkoxy group; R₅ is selected from the group consisting of a hydrogen, a hydroxyl, a carbonyl, an alkoxy and a bond; R₆ is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; R7 is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; and R₈ is selected from the group consisting of a hydrogen, a hydroxyl group, a carbonyl, an alkoxy group and a bond.

In another aspect, the sulfatide has following chemical structure:

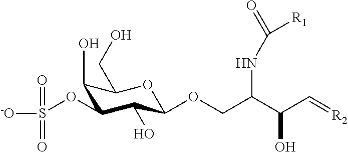

wherein R₁ is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; and R₂ is selected from the group consisting of a hydrogen, a hydroxyl group, a carbonyl, an alkoxy group and a bond.

In another aspect, the sulfatide can have the following chemical structure:

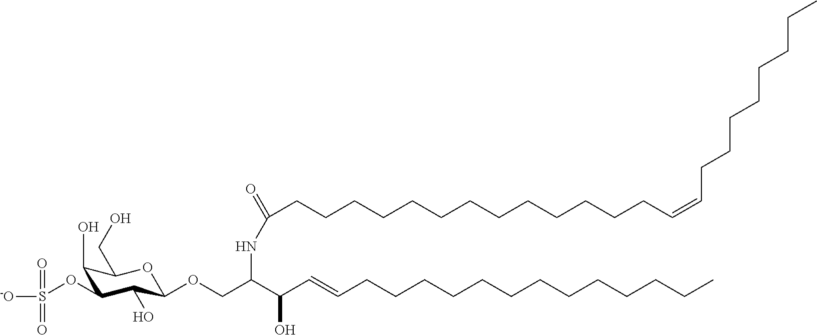

In yet another aspect, the sulfatide is: (2S,3R,4E)-N-nervonic-1[-D-(3-sulfate)-galactopyranosyl]-2-amino-octadecene-3-ol.

In still another aspect, the autoimmune disease can be, for example, systemic lupus erythematsosus, AIDS, Alzheimer's disease, rheumatoid arthritis, insulin dependent diabetes mellitus, autoimmune hepatitis, asthma or celiac disease.

In another aspect, the sulfatide can be administered by one or more of the following routes: intravenous, intraperitoneal, inhalation, intramuscular, subcutaneous and oral.

Another embodiment relates to a method of treating a patient with symptoms of an autoimmune disease comprising administering an amount of a sulfatide effective to reduce said symptoms, wherein the sulfatide has the following chemical structure:

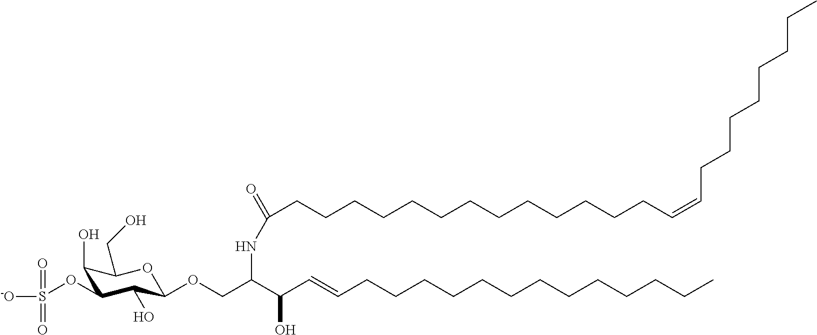

In one aspect, the autoimmune disease can be, for example, multiple sclerosis, systemic lupus erythematsosus, AIDS, Alzheimer's disease, rheumatoid arthritis, insulin dependent diabetes mellitus, autoimmune hepatitis, asthma or celiac disease.

In another aspect, the autoimmune disease is multiple sclerosis.

Another embodiment relates to a method of treating the indications of an autoimmune disease in a patient comprising administering to said patient a therapeutically effective amount of a sulfatide, wherein the autoimmune disease is not multiple sclerosis.

In one aspect, the sulfatide has following chemical structure:

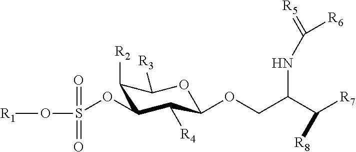

wherein R₁ is selected from the group consisting of a bond, a hydrogen, a C₁ to C₃₀ alkyl, C₁ to C₃₀ substituted alkyl, a C₁ to C₃₀ alkenyl, a C₁ to C₃₀ substituted alkenyl and a C₅ to C₁₂ sugar; R₂ is selected from the group consisting of a hydrogen, a hydroxy group, a methoxy group, and an alkoxy group; R₃ is selected from the group consisting of a hydrogen, a hydroxy group, a methoxy group, an ethoxy group, and an alkoxy group; R₄ is selected from the group consisting of a hydrogen, a hydroxy group and an alkoxy group; R₅ is selected from the group consisting of a hydrogen, a hydroxyl, a carbonyl, an alkoxy and a bond; R₆ is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; R7 is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; and R₈ is selected from the group consisting of a hydrogen, a hydroxyl group, a carbonyl, an alkoxy group and a bond.

In another aspect, the sulfatide has the following chemical structure:

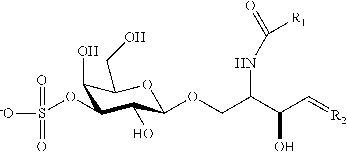

wherein R₁ is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; and R₂ is selected from the group consisting of a hydrogen, a hydroxyl group, a carbonyl, an alkoxy group and a bond.

In another aspect, the sulfatide is (2S,3R,4E)-N-nervonic-1-[-D-(3-sulfate)-galactopyranosyl]-2-amino-octadecene-3-ol.

In yet another aspect, the sulfatide has the following chemical structure:

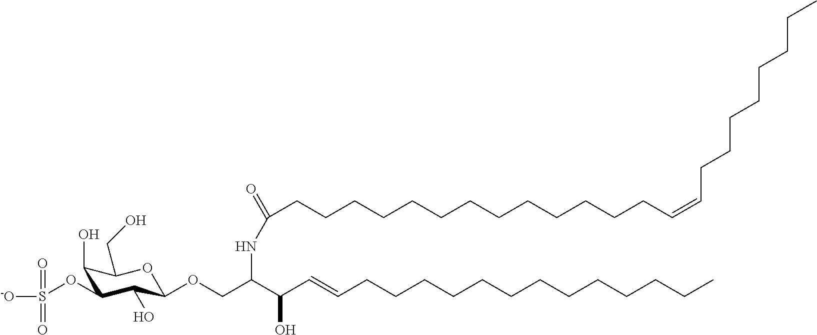

In yet another aspect, the autoimmune disease can be, for example, systemic lupus erythematsosus, AIDS, Alzheimer's disease, rheumatoid arthritis, insulin dependent diabetes mellitus, autoimmune hepatitis, asthma or celiac disease.

In still another aspect, the autoimmune disease can be multiple sclerosis.

In another aspect, the autoimmune disease is AIDS.

In another aspect, the sulfatide can be administered by one or more of the following routes: intravenous, intraperitoneal, inhalation, intramuscular, subcutaneous and oral.

Another embodiment relates to a method of treating or preventing the symptoms of an autoimmune disease in a mammal, comprising the step of administering to said mammal a therapeutically effective amount of a sulfatide formulated in a pharmaceutically acceptable vehicle, wherein the autoimmune disease is not multiple sclerosis.

In one aspect, the mammal is a human.

In another aspect, the sulfatide has following chemical structure:

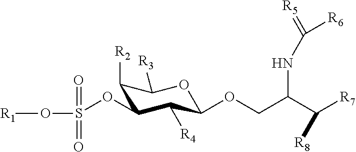

wherein R₁ is selected from the group consisting of a bond, a hydrogen, a C₁ to C₃₀ alkyl, C₁ to C₃₀ substituted alkyl, a C₁ to C₃₀ alkenyl, a C₁ to C₃₀ substituted alkenyl and a C₅ to C₁₂ sugar; R₂ is selected from the group consisting of a hydrogen, a hydroxy group, a methoxy group, and an alkoxy group; R₃ is selected from the group consisting of a hydrogen, a hydroxy group, a methoxy group, an ethoxy group, and an alkoxy group; R₄ is selected from the group consisting of a hydrogen, a hydroxy group and an alkoxy group; R₅ is selected from the group consisting of a hydrogen, a hydroxyl, a carbonyl, an alkoxy and a bond; R₆ is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; R7 is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; and R₈ is selected from the group consisting of a hydrogen, a hydroxyl group, a carbonyl, an alkoxy group and a bond.

In yet another aspect, the sulfatide has following chemical structure:

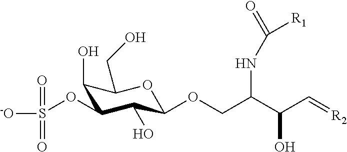

wherein R₁ is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; and R₂ is selected from the group consisting of a hydrogen, a hydroxyl group, a carbonyl, an alkoxy group and a bond.

In still another aspect, the sulfatide is (2S,3R,4E)-N-nervonic-1-[-D-(3-sulfate)-galactopyranosyl]-2-amino-octadecene-3-ol.

In another aspect, the sulfatide has the following chemical formula:

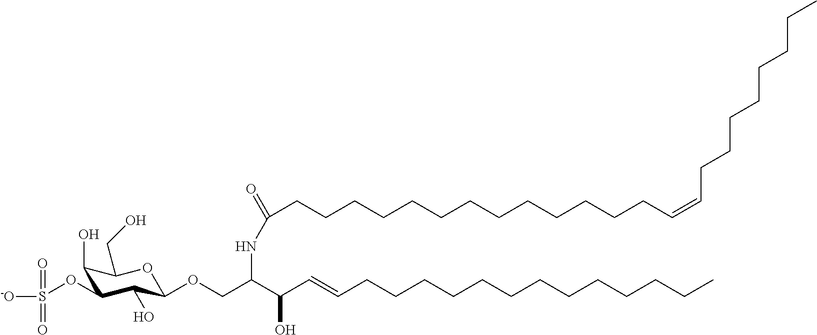

In another aspect, the autoimmune disease is can be, for example, systemic lupus erythematsosus, AIDS, Alzheimer's disease, rheumatoid arthritis, insulin dependent diabetes mellitus, autoimmune hepatitis, asthma and celiac disease.

In yet another aspect, the autoimmune disease is multiple sclerosis.

In still another aspect, the autoimmune disease is AIDS.

In another aspect, the autoimmune disease is asthma.

In another aspect, the sulfatide is administered by one or more of the following routes: intravenous, intraperitoneal, inhalation, intramuscular, subcutaneous and oral.

One embodiment relates to a method for evaluating the effectiveness of a sulfatide in preventing ischemia and reperfusion injury comprising: administering an effective amount of the sulfatide to a mouse; inducing a hepatic ischemia/reperfusion injury; and assessing a liver sample taken from the mouse after 6 hours of reperfusion for markers of ischemia and reperfusion injury, wherein the marker of reperfusion injury is IFN-γ secretion.

One embodiment relates to a method for treating or preventing at least one of hepatic ischemia and hepatic reperfusion injury associated with a surgical procedure in a patient comprising administering an effective amount of sulfatide to the patient prior to undergoing the surgical procedure.

Another embodiment relates to a method for treating or preventing at least one of hepatic ischemia and hepatic reperfusion injury associated with a surgical procedure in a patient comprising administering an effective amount of sulfatide to the patient prior to undergoing the surgical procedure, wherein the sulfatide is administered from about 1 hour to about 24 hours prior to the surgical procedure.

Another embodiment relates to a method for treating or preventing at least one of hepatic ischemia and hepatic reperfusion injury associated with a surgical procedure in a patient comprising administering an effective amount of sulfatide to the patient prior to undergoing the surgical procedure, wherein the sulfatide is administered from about 2 hours to about 8 hours prior to the surgical procedure.

Another embodiment relates to a method for treating or preventing at least one of hepatic ischemia and hepatic reperfusion injury associated with a surgical procedure in a patient comprising administering an effective amount of sulfatide to the patient prior to undergoing the surgical procedure, wherein the sulfatide is administered about 6 hours prior to the surgical procedure.

Another embodiment relates to a method for treating or preventing at least one of hepatic ischemia and hepatic reperfusion injury associated with a surgical procedure in a patient comprising administering an effective amount of sulfatide to the patient prior to undergoing the surgical procedure, wherein the amount of sulfatide administered is about 1μ gram/kg of body weight.

Another embodiment relates to a method for treating or preventing at least one of hepatic ischemia and hepatic reperfusion injury associated with a surgical procedure in a patient comprising administering an effective amount of sulfatide to the patient prior to undergoing the surgical procedure, wherein the sulfatide has following chemical structure:

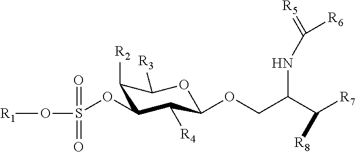

wherein R1 is selected from the group consisting of a bond, a hydrogen, a C₁ to C₃₀ alkyl, C₁ to C₃₀ substituted alkyl, a C₁ to C₃₀ alkenyl, a C₁ to C₃₀ substituted alkenyl and a C₅ to C₁₂ sugar; R₂ is selected from the group consisting of a hydrogen, a hydroxy group, a methoxy group, and an alkoxy group; R₃ is selected from the group consisting of a hydrogen, a hydroxy group, a methoxy group, an ethoxy group, and an alkoxy group; R₄ is selected from the group consisting of a hydrogen, a hydroxy group and an alkoxy group; R₅ is selected from the group consisting of a hydrogen, a hydroxyl, a carbonyl, an alkoxy and a bond; R₆ is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; R₇ is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; and R₈ is selected from the group consisting of a hydrogen, a hydroxyl group, a carbonyl, an alkoxy group and a bond.

Another embodiment relates to a method for treating or preventing at least one of hepatic ischemia and hepatic reperfusion injury associated with a surgical procedure in a patient comprising administering an effective amount of sulfatide to the patient prior to undergoing the surgical procedure, wherein the sulfatide has following chemical structure:

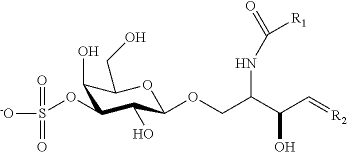

wherein R₁ is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; and R₂ is selected from the group consisting of a hydrogen, a hydroxyl group, a carbonyl, an alkoxy group and a bond.

Another embodiment relates to a method for treating or preventing at least one of hepatic ischemia and hepatic reperfusion injury associated with a surgical procedure in a patient comprising administering an effective amount of sulfatide to the patient prior to undergoing the surgical procedure, wherein the sulfatide has the following chemical structure:

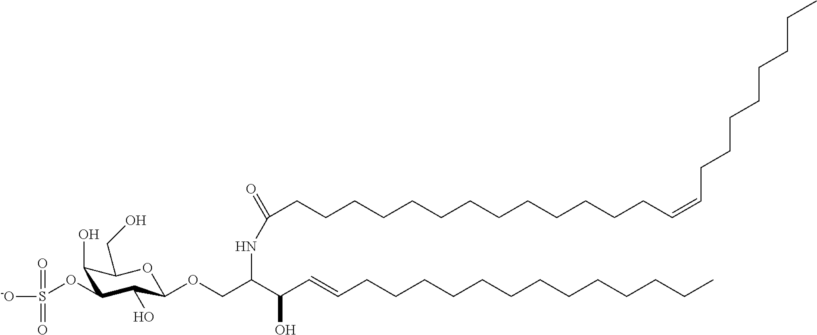

Another embodiment relates to a method for treating or preventing at least one of hepatic ischemia and hepatic reperfusion injury associated with a surgical procedure in a patient comprising administering an effective amount of sulfatide to the patient prior to undergoing the surgical procedure and further comprising administering sulfatide during the surgical procedure and/or after the surgical procedure.

One embodiment relates to a method for preventing hepatic ischemia and reperfusion injury comprising administering an agent which inhibits the activity of type I NKT by activating type II NKT.

Another embodiment relates to a method for preventing hepatic ischemia and reperfusion injury comprising administering an agent which inhibits the activity of type I NKT by activating type II NKT by administering a sulfatide.

Another embodiment relates to a method for preventing hepatic ischemia and reperfusion injury comprising administering an agent which inhibits the activity of type I NKT by activating type II NKT by administering a sulfatide 6 hours prior to surgery.

Another embodiment relates to a method for preventing hepatic ischemia and reperfusion injury comprising administering an agent which inhibits the activity of type I NKT by activating type II NKT by administering a sulfatide having the following chemical structure:

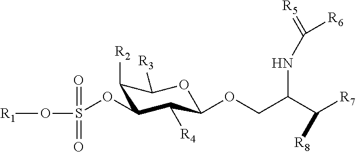

wherein R₁ is selected from the group consisting of a bond, a hydrogen, a C₁ to C₃₀ alkyl, C₁ to C₃₀ substituted alkyl, a C₁ to C₃₀ alkenyl, a C₁ to C₃₀ substituted alkenyl and a C₅ to C₁₂ sugar; R₂ is selected from the group consisting of a hydrogen, a hydroxy group, a methoxy group, and an alkoxy group; R₃ is selected from the group consisting of a hydrogen, a hydroxy group, a methoxy group, an ethoxy group, and an alkoxy group; R₄ is selected from the group consisting of a hydrogen, a hydroxy group and an alkoxy group; R₅ is selected from the group consisting of a hydrogen, a hydroxyl, a carbonyl, an alkoxy and a bond; R₆ is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; R₇ is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; and R₈ is selected from the group consisting of a hydrogen, a hydroxyl group, a carbonyl, an alkoxy group and a bond.

Another embodiment relates to a method for preventing hepatic ischemia and reperfusion injury comprising administering an agent which inhibits the activity of type I NKT by activating type II NKT by administering a sulfatide having the following chemical structure:

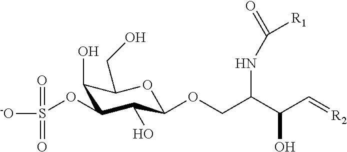

wherein R₁ is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; and R₂ is selected from the group consisting of a hydrogen, a hydroxyl group, a carbonyl, an alkoxy group and a bond.

Another embodiment relates to a method for preventing hepatic ischemia and reperfusion injury comprising administering an agent which inhibits the activity of type I NKT by activating type II NKT by administering a sulfatide having the following chemical structure:

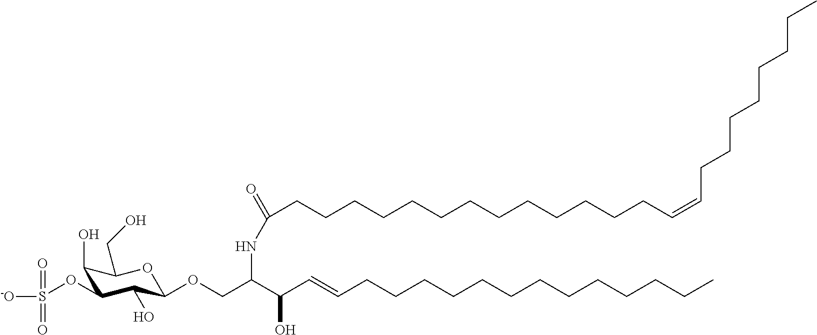

One embodiment relates to a method for evaluating the effectiveness of a sulfatide in preventing ischemia and reperfusion injury comprising: administering an effective amount of the sulfatide to a subject; inducing a hepatic ischemia/reperfusion injury; and assessing a liver sample taken from the subject after 6 hours of reperfusion for markers of ischemia and reperfusion injury.

Another embodiment relates to a method for evaluating the effectiveness of a sulfatide in preventing ischemia and reperfusion injury comprising: administering an effective amount of the sulfatide to a subject; inducing a hepatic ischemia/reperfusion injury; and assessing a liver sample taken from the subject after 6 hours of reperfusion for markers of ischemia and reperfusion injury, wherein the marker of reperfusion injury is elevated levels of Alanine aminotransferase (ALT) enzyme.

Another embodiment relates to a method for evaluating the effectiveness of a sulfatide in preventing ischemia and reperfusion injury comprising: administering an effective amount of the sulfatide to a subject; inducing a hepatic ischemia/reperfusion injury; and assessing a liver sample taken from the subject after 6 hours of reperfusion for markers of ischemia and reperfusion injury, wherein the marker of reperfusion injury is IFN-γ secretion.

Another embodiment relates to a method for evaluating the effectiveness of a sulfatide in preventing ischemia and reperfusion injury comprising: administering an effective amount of the sulfatide to a subject; inducing a hepatic ischemia/reperfusion injury; and assessing a liver sample taken from the subject after 6 hours of reperfusion for markers of ischemia and reperfusion injury, wherein the sulfatide has following chemical structure:

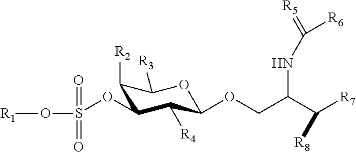

wherein R1 is selected from the group consisting of a bond, a hydrogen, a C₁ to C₃₀ alkyl, C₁ to C₃₀ substituted alkyl, a C₁ to C₃₀ alkenyl, a C₁ to C₃₀ substituted alkenyl and a C₅ to C₁₂ sugar; R₂ is selected from the group consisting of a hydrogen, a hydroxy group, a methoxy group, and an alkoxy group; R₃ is selected from the group consisting of a hydrogen, a hydroxy group, a methoxy group, an ethoxy group, and an alkoxy group; R₄ is selected from the group consisting of a hydrogen, a hydroxy group and an alkoxy group; R₅ is selected from the group consisting of a hydrogen, a hydroxyl, a carbonyl, an alkoxy and a bond; R₆ is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; R₇ is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; and R₈ is selected from the group consisting of a hydrogen, a hydroxyl group, a carbonyl, an alkoxy group and a bond.

One embodiment relates to a method of treating or preventing ischemia or reperfusion injury in a patient comprising administering an effective amount of sulfatide to the patient in need thereof.

Another embodiment relates to a method of treating or preventing ischemia or reperfusion injury in a patient comprising administering an effective amount of sulfatide to the patient wherein the patient is a surgical patient.

Another embodiment relates to a method of treating or preventing ischemia or reperfusion injury in a patient comprising administering an effective amount of sulfatide to the patient in need thereof, wherein the ischemia or reperfusion injury is associated with at least one of myocardial infarction, arteriosclerosis, stroke, septic shock, traumatic shock, vascular interventional procedures, angioplasty, abdominal surgery, abdominoplasty, adenoidectomy, amputation, appendectomy, arthrodesis, arthroplasty, sickle cell anemia, brain surgery, cesarean section, cholecystectomy, colon resection, colostomy, corneal transplantation, discectomy, endarterectomy, gastrectomy, grafting of skin or other tissues, heart transplantation, heart surgery, hemicorporectomy, hemorrhoidectomy, hepatectomy, hernia repair, hysterectomy, kidney transplantation, laminectomy, laryngectomy, crush injury, myocardial infarction, stroke, brain trauma, hepatic surgery, hepatic injury, renal surgery, renal injury, lumpectomy, liver transplantation, lung transplantation, kidney transplantation, mammoplasty, mastectomy, mastoidectomy, myotomy, nephrectomy, nissen fundoplication, oophorectomy, orchidectomy, orthopedic surgery, parathyroidectomy, plastic surgery, penectomy, phalloplasty, pneumonectomy, prostatectomy, radiosurgery, rotationplasty, splenectomy, stapedectomy, thoracotomy, thrombectomy, thymectomy, thyroidectomy, tonsillectomy, ulnar collateral ligament reconstruction, vaginectomy, vasectomy, cardiac bypass, cardiac artery bypass, graft surgery and organ transplantation.

Another embodiment relates to a method of treating or preventing ischemia or reperfusion injury in a patient comprising administering an effective amount of sulfatide to the patient in need thereof, wherein the ischemia or reperfusion injury is related to liver transplantation or liver injury.

Another embodiment relates to a method of treating or preventing ischemia or reperfusion injury in a patient comprising administering an effective amount of sulfatide to the patient in need thereof, wherein the administering is intraperitoneal.

Another embodiment relates to a method of treating or preventing ischemia or reperfusion injury in a patient comprising administering an effective amount of sulfatide to the patient in need thereof, wherein the administering is intravenous.

Another embodiment relates to a method of treating or preventing ischemia or reperfusion injury in a patient comprising administering an effective amount of sulfatide to the patient in need thereof, wherein the sulfatide is administered prior to a surgical procedure.

Another embodiment relates to a method of treating or preventing ischemia or reperfusion injury in a patient comprising administering an effective amount of sulfatide to the patient in need thereof, wherein the sulfatide is administered during or after a surgical procedure.

Another embodiment relates to a method of treating or preventing ischemia or reperfusion injury in a patient comprising administering an effective amount of sulfatide to the patient in need thereof, wherein the sulfatide is administered The method of Claim 20, wherein the sulfatide has following chemical structure:

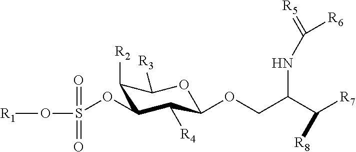

wherein R1 is selected from the group consisting of a bond, a hydrogen, a C₁ to C₃₀ alkyl, C₁ to C₃₀ substituted alkyl, a C₁ to C₃₀ alkenyl, a C₁ to C₃₀ substituted alkenyl and a C₅ to C₁₂ sugar; R₂ is selected from the group consisting of a hydrogen, a hydroxy group, a methoxy group, and an alkoxy group; R₃ is selected from the group consisting of a hydrogen, a hydroxy group, a methoxy group, an ethoxy group, and an alkoxy group; R₄ is selected from the group consisting of a hydrogen, a hydroxy group and an alkoxy group; R₅ is selected from the group consisting of a hydrogen, a hydroxyl, a carbonyl, an alkoxy and a bond; R₆ is selected from the group consisting of a C₁ to C₄₀ alkyl, a C1 to C₄₀ substituted alkyl, a C₁ to C₄₀ alkenyl, a C1 to C₄₀ substituted alkenyl and a C₁ to C₄₀ alkynl; R₇ is selected from the group consisting of a C₁ to C₄₀ alkyl, a C1 to C₄₀ substituted alkyl, a C₁ to C₄₀ alkenyl, a C₁ to C₄₀ substituted alkenyl and a C₁ to C₄₀ alkynl; and R₈ is selected from the group consisting of a hydrogen, a hydroxyl group, a carbonyl, an alkoxy group and a bond.

Another embodiment relates to a method of treating or preventing ischemia or reperfusion injury in a patient comprising administering an effective amount of sulfatide to the patient in need thereof, wherein the sulfatide is administered The method of Claim 20, wherein the sulfatide has following chemical structure:

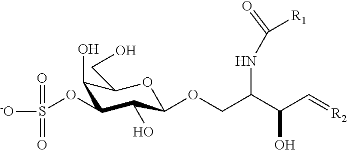

wherein R₁ is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; and R₂ is selected from the group consisting of a hydrogen, a hydroxyl group, a carbonyl, an alkoxy group and a bond.

Another embodiment relates to a method of treating or preventing ischemia or reperfusion injury in a patient comprising administering an effective amount of sulfatide to the patient in need thereof, wherein the sulfatide is administered The method of Claim 20, wherein the sulfatide has following chemical structure:

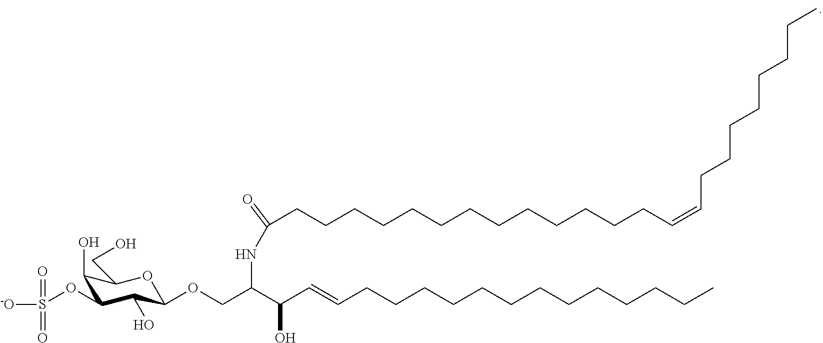

### BRIEF DESCRIPTION OF THE DRAWINGS

FIG. 1 shows that treatment of mice with sulfatide reverses ongoing chronic EAE in C57.BL/6J mice. Mice were injected intraperitoneally with 20 μg of bovine brain sulfatide (▪) or with PBS/vehicle alone (Δ) at the onset of disease (↑).

FIG. 2 shows that treatment of mice with sulfatide reverses ongoing chronic-relapsing EAE in SJL/J mice. Mice were injected intraperitoneally with 20 μg of bovine brain sulfatide (▪) or with PBS/vehicle alone (Δ) at the onset of disease (left ↑) and fourteen days later (right ↑).

FIG. 3 shows that treatment of mice with cis-teracosenoyl sulfatide reverses ongoing chronic-relapsing EAE in SJL/J mice. Mice were injected intraperitoneally with 20 μg of cis-teracosenoyl sulfatide () or with PBS/vehicle alone (Δ) at the onset of disease (↑).

FIG. 4 shows that administration of sulfatide in nonobese diabetic (NOD) mice prevents diabetes. Female and male NOD mice were treated with bovine brain sulfatide, (▴) and () or with control lipid (Mono GM-1), (Δ) and (◯).

FIG. 5 shows that serum enzymes, alanine amino transferase (ALT) (top plot), and aspartate amino transferase (AST) (bottom plot) levels significantly decreased in Con A+bovine brain sulfatide (◯) injected mice in comparison to the Con A () injected mice.

FIG. 6 shows hematoxylin and eosin stained liver sections demonstrating markedly improved hepatic histology in sulfatide-treatment mice (second panel) relative to mice in the control group (first panel) at indicated time points. Second panel includes a control liver section following only bovine brain sulfatide injection.

FIG. 7 shows a complete protection from gross morphological liver damage from hepatitis following sulfatide treatment. Liver morphology of Con A injected mice (second panel), Con A+bovine brain sulfatide injected mice (third panel) and PBS or 24 hour sulfatide injected mice as control samples (first panel).

FIG. 8 shows that administration of bovine brain sulfatide in SCID-hu mice drastically lowers HIV-1 infection as well as HIV-1 replication.

FIG. 9 shows that administration of bovine brain sulfatide in SCID-hu mice maintains the multilineage colony forming activity of human thymocytes during HIV-1 infection.

FIG. 10 shows that administration of cis-tetracosenoyl sulfatide reduces the mean disease score in chronic-relapsing/remitting EAE in SJL/J (H-2s) mice when injected 12, 23, 33, and 45 days after disease onset. Mice were injected intraperitoneally with cis-tetracosenoyl sulfatide (▪) or with PBS/vehicle alone (Δ).

FIG. 11 shows that administration of cis-tetracosenoyl sulfatide reduces the mean disease score in chronic-relapsing/remitting EAE in SJL/J (H-2s) mice when injected 12, 23, and 33 days after disease onset. Mice were injected intraperitoneally with cis-tetracosenoyl sulfatide (▪) or with PBS/vehicle alone (Δ).

FIG. 12 shows that administration of cis-tetracosenoyl sulfatide reduces the mean disease score in chronic-relapsing/remitting EAE in SJL/J (H-2s) mice when injected 19 and 35 days after disease onset. Mice were injected intraperitoneally with cis-tetracosenoyl sulfatide (▪) or with PBS/vehicle alone (Δ).

FIG. 13 shows that administration of cis-tetracosenoyl sulfatide reduces mean disease score in the C57.BL/6J mouse model of chronic EAE. Mice were injected intraperitoneally with cis-tetracosenoyl sulfatide (▪) or with PBS/vehicle alone (Δ) on days 0 and 21 after MOG35-55 immunization.

FIG. 14 shows that administration of cis-tetracosenoyl sulfatide reduces the peak in mean disease score in monophasic disease in B10.PL (H-2u) mice. Mice were injected intraperitoneally with cis-tetracosenoyl sulfatide (▪) or with PBS/vehicle alone (Δ) on days 0 and 13 after Ac1-9 immunization.

FIG. 15 shows that hepatic necroses associated with ischemia and reperfusion injury are reduced in the absence of type I NKT cells or following activation of type II NKT cells. The panels show representative (of at least 3 mice) H&E staining of tissue from cephalad liver lobes at 100× magnification. From left to right, the panels show tissue from untreated WT, untreated Jα18^{-/-}, sulfatide-treated WT, and sulfatide-treated Jα18^{-/-} mice. The top row shows liver tissue taken from mice subjected to 90 min of hepatic ischemia followed by 24 hrs of reperfusion (IRI) and the bottom row shows liver tissue taken from mice subjected to sham surgery (sham).

FIG. 16 shows in the absence of type I NKT cells or following activation of type II NKT cells the levels of serum ALT are reduced. The bar graph depicts serum ALT levels following 90 minutes of ischemia and 6 hrs of reperfusion (IRI) or sham surgery (sham) for untreated WT, sulfatide-treated WT, untreated Jα18^{-/-}, and sulfatide-treated Jα18^{-/-} mice. Values are mean±SEM; *p<0.05.

FIG. 17 shows that sulfatide administration prior to ischemia and reperfusion injury induction significantly inhibits IFN-γ secretion by type I NKT cells. The Upper panels show Tri-color flow cytometric analysis of IFN-γ⁺ cells in αGalCer/CD1d-tetramer⁺ cells. The solid line indicates IFN-γ and the dashed line indicates isotype. Numbers above brackets indicate percent positive cells. The Lower panel shows a bar graph summarizing the cytometric analysis. Values are mean±SEM. *p<0.01.

FIG. 18 shows bar graphs summarizing the results of flow cytometric analysis of the indicated cell populations among mononuclear cells MNCs or leukocytes (for CD11b⁺ cells) from cephalad liver lobes following 90 min of ischemia and 6 hrs of reperfusion (IRI) or sham surgery. Changes in the proportion (left panels) and absolute number (right panels) of hepatic NK (NK1.1⁺TCRβ⁻) cells are shown for untreated WT mice (WT), Jα18^{-/-} mice (Jα18^{-/-}), and sulfatide-treated WT mice (Sulfatide). Values are mean±SEM. *p<0.05. n.d.=not done.

FIG. 19 shows flow cytometric analysis of CD11b⁺Gr-1⁺ subsets among leukocytes from cephalad liver lobes following 90 min of ischemia and 6 hrs of reperfusion (IRI) or sham surgery in WT or Jα18^{-/-} mice (3-5 mice/group) and in WT mice treated with sulfatide 3 hrs earlier (2-3 mice/group). The upper panels show the Gr-1^{high} and Gr-1^{int} populations. Numbers next to boxes indicate percent positive cells among liver leukocytes. The lower panels show summarizing bar graphs depicting the % (left) and absolute numbers (right) of the Gr-1^{high} and Gr-1^{int} populations. Values are mean±SEM. *p<0.005.

FIG. 20 shows a diagram of a proposed model of Type I and Type II NKT cell activity in hepatic ischemia and reperfusion injury.

### DETAILED DESCRIPTION

NKT cells have the ability to regulate the activity of other cells that significantly contribute to inflammation of tissue and the associated cellular damage. Most NKT cells recognize lipid antigens presented in the context of the monomorphic MEC class I-like molecule CD1d. CD1d-restricted NKT cells are categorized into type I and type II. Type I or invariant NKT cells express a semi-invariant TCR encoded by the Vα14-Jα18 gene segments in mice, are strongly reactive with the marine sponge-derived glycolipid α-galactosyl ceramide (αGalCer), and are identified by αGalCer/CD1d-tetramers in flow cytometry. Type I or invariant NKT also recognize bacterial-derived lipids and a self-glycolipid, isoglobotrihexosyl ceramide (iGb3).

Type II NKT cells are regulatory cells that can modulate the activity of several other cell subsets, including type I NKT cells. Some embodiments relate to a major subset of type II NKT cells which are reactive to sulfatides. Some embodiments relate to a major subset of type II NKT cells which are reactive to the self-glycolipid, 3-sulfated β-galactosyl ceramide, an example of a sulfatide. These type II NKT cells which are reactive to sulfatide can be identified using sulfatide/CD1d-tetramers in flow cytometry. The present embodiments relate to the regulation of type I NKT cells by activated sulfatide-reactive type II NKT cells. Several embodiments relate to the regulatory role of activated sulfatide-reactive type II NKT cells on type I NKT cells in mediating protection from autoimmune disease and suppression of anti-tumor immunity.

The present embodiments are related to treatments for ischemia, reperfusion injury and a wide variety of autoimmune or immune related diseases or disorders including, for example, multiple sclerosis, systemic lupus erythematsosus, AIDS, Alzheimer's disease, rheumatoid arthritis, insulin dependent diabetes mellitus, autoimmune hepatitis, asthma, and celiac disease.

Some embodiments relate to methods for treating such reperfusion injury, autoimmune or immune related diseases or disorders with the administration of sulfatides. More specifically, some embodiments relate to methods of treating reperfusion injury, autoimmune or immune related diseases or disorders by administering an amount of a sulfatide to the body of a patient effective to reduce or prevent the symptoms of the autoimmune or immune related disease or disorder. In preferred embodiments the sulfatide has the following chemical formula I:

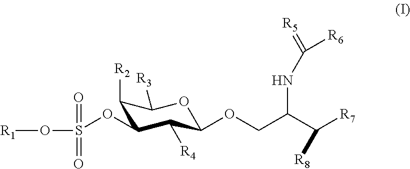

wherein R₁ can be a bond, a hydrogen, a C₁ to C₃₀ alkyl, a C₁ to C₃₀ substituted alkyl, a C₁ to C₃₀ alkenyl, a C₁ to C₃₀ substituted alkenyl or a C₅ to C₁₂ sugar; R₂ can be a hydrogen, a hydroxy group, a methoxy group, or an alkoxy group; R₃ can be a hydrogen, a hydroxy group, a methoxy group, an ethoxy group, or an alkoxy group; R₄ can be a hydrogen, a hydroxy group or an alkoxy group; R₅ can be a hydrogen, a hydroxy group, a carbonyl, an alkoxy group or a bond; R₆ can be a C₁ to C₄₀ alkyl, a C₁ to C₄₀ substituted alkyl, a C₁ to C₄₀ alkenyl, a C₁ to C₄₀ substituted alkenyl, or a C₁ to C₄₀ alkynl; R7 can be a C₁ to C₄₀ alkyl, a C₁ to C₄₀ substituted alkyl, a C₁ to C₄₀ alkenyl, a C₁ to C₄₀ substituted alkenyl, or a C₁ to C₄₀ alkynl; and R₈ can be a hydrogen, a hydroxy group, a carbonyl, an alkoxy group or a bond.

In other embodiments, the sulfatide has the following chemical formula II:

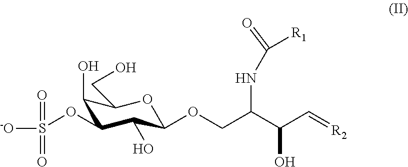

wherein R₁ is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; and R₂ is selected from the group consisting of a hydrogen, a hydroxyl group, a carbonyl, an alkoxy group and a bond.

As used herein, the term “alkyl” means any unbranched or branched, saturated hydrocarbon. The term “substituted alkyl” means any unbranched or branched, substituted saturated hydrocarbon. Cyclic compounds, both cyclic hydrocarbons and cyclic compounds having heteroatoms, are within the meaning of “alkyl.”

As used herein, the term “substituted” means any substitution of a hydrogen atom with a functional group.

As used herein, the term “functional group” has its common definition, and refers to chemical moieties preferably selected from the group consisting of a halogen atom, C₁-C₂₀ alkyl, substituted C₁-C₂₀ alkyl, perhalogenated alkyl, cyloalkyl, substituted cycloalkyl, aryl, substituted aryl, benzyl, heteroaryl, substituted heteroaryl, cyano, and nitro.

As used herein, the terms “halogen” and “halogen atom” refer to any one of the radio-stable atoms of column 17 of the Periodic Table of the Elements, preferably fluorine, chlorine, bromine, or iodine, with fluorine and chlorine being particularly preferred.

As used herein, the term “alkenyl” means any unbranched or branched, substituted or unsubstituted, unsaturated hydrocarbon. The term “substituted alkenyl” means any unbranched or branched, substituted unsaturated hydrocarbon, substituted with one or more functional groups, with unbranched C₂-C₆ alkenyl secondary amines, substituted C₂-C₆ secondary alkenyl amines, and unbranched C₂-C₆ alkenyl tertiary amines being within the definition of “substituted alkyl.” Cyclic compounds, both unsaturated cyclic hydrocarbons and cyclic compounds having heteroatoms, are within the meaning of “alkenyl.”

As used herein, the term “alkoxy” refers to any unbranched, or branched, substituted or unsubstituted, saturated or unsaturated ether.

As used herein, the term “sulfatide” retains its general accustomed meaning and refers to a cerebroside sulfuric ester containing one or more sulfate groups in the sugar portion of the molecule.

As used herein, the term “cerebroside” refers to any lipid compound containing a sugar, and generally of the type normally found in the brain and nerve tissue.

As used herein the term “an effective amount” of an agent is the amount sufficient to treat, inhibit, or prevent ischemia and/or reperfusion injury associated with indications and conditions including, but not limited to, myocardial infarction, arteriosclerosis. stroke, septic shock, traumatic shock, and associated with surgical procedures such as vascular interventional procedures including angioplasty, surgery that involves restriction of blood supply to an organ or tissue, abdominal surgery, abdominoplasty, adenoidectomy, amputation, angioplasty, appendectomy, arthrodesis, arthroplasty, brain surgery, cesarean section, cholecystectomy, colon resection, colostomy, corneal transplantation, discectomy, endarterectomy, gastrectomy, grafting of skin or other tissues, heart transplantation, liver transplantation, heart surgery hemicorporectomy, hemorrhoidectomy, hepatectomy, hernia repair, hysterectomy, kidney transplantation, laminectomy, laryngectomy, lumpectomy, lung transplantation, mammoplasty, mastectomy, mastoidectomy, myotomy, nephrectomy, nissen fundoplication, oophorectomy, orchidectomy, orthopedic surgery, parathyroidectomy, penectomy, phalloplasty, pneumonectomy, prostatectomy, radiosurgery, rotationplasty, splenectomy, stapedectomy, thoracotomy, thrombectomy, thymectomy, thyroidectomy, tonsillectomy, ulnar collateral ligament reconstruction, vaginectomy, vasectomy and any surgery involving cardiac bypass, cardiac artery bypass graft surgery and organ transplantation.

The compounds of formula (I), (II) and (III) may be in the form of pharmaceutically acceptable nontoxic salts thereof. Salts of formula (I), (II) and (III) include acid added salts, such as salts with inorganic acids (e.g., hydrochloric acid, sulfuric acid, nitric acid and phosphoric acid) or with organic acids (e.g., acetic acid, propionic acid, maleic acid, oleic acid, palmitic acid, citric acid, succinic acid, tartaric acid, fumaric acid, glutamic acid, pantothenic acid, laurylsulfonic acid, methanesulfonic acid and phthalic acid).

The compounds of formula (I), (II) and (III) may be in the form of solvates thereof (e.g., hydrates).

The compounds of formula (I), (II) and (III) can be produced by any purposive method to synthesize sulfatides.

The compounds of formulas (I), (II) and (III) can also be isolated from natural products (e.g., biological organisms) and purified by column chromatography or the like.

In one embodiment, the sulfatide has the chemical formula: (2S,3R,4E)-N-nervonic-1-[-D-(3-sulfate)-galactopyranosyl]-2-amino-octadecene-3-ol. This chemical formula is also referred to as cis-tetracosenoyl sulfatide.

In another embodiment, the sulfatide has the following chemical structure:

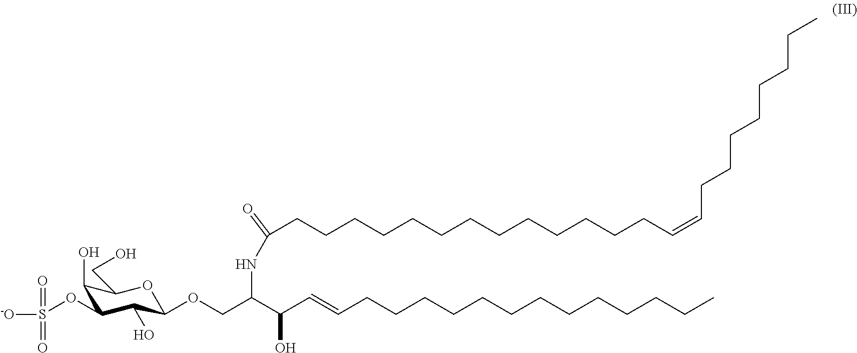

In some embodiments, the sulfatide can be, for example, bovine brain-derived sulfatide which is a mixture of about 20 different species obtained from Sigma Inc. (Chicago, Ill., USA). In other embodiments, the sulfatide is semisynthetic and is a single species of sulfatide, for example, cis-tetracosenoyl sulfatide or lysosulfatide obtained from Maitreya Inc, (Pleasant Gap, Pa., USA). In still other embodiments, the sulfatide can be a totally synthetic sulfatide.

Another embodiment is related to a method of treating the various indications of autoimmune or immune related diseases or disorders. In particular, one aspect of the present embodiment is related to a method of treating a patient suffering from symptoms of an autoimmune or immune related disease or disorder, such as, for example, multiple sclerosis, systemic lupus erythematsosus, AIDS, Alzheimer's disease, rheumatoid arthritis, insulin dependent diabetes mellitus, autoimmune hepatitis, asthma, and celiac disease.

Some embodiments relate to a method of treating asthma in a patient. Bronchial asthma is associated with an inflammatory process in the lungs that is characterized by the presence in the airways of large numbers of cytokines-secreting CD4+ T cells. CD4 antigen is expressed not only by class II major histocompatibility complex (MHC)-restricted CD4+ T cells, but also by CD1-restricted NK T cells. These cells can be categorized into 2 subsets based on those using invariant receptors, such as invariant NK T cells (“iNK T cells”) and those using variable receptors, such as non-invariant NK T cells (“non-iNK T cells”). Mouse models of allergic asthma have shown that NK T cells are required for the development of allergen-induced airway hyperreactivity.

Studies have also shown that a major percentage of the pulmonary CD4+CD3+ cells in patients with moderate-to-severe persistent asthma were iNK T cells. However, this was not the case in the lungs of patients with sarcoidosis. Since these iNK T cells constitute only a minor population (around 0.1%) of the CD4+ T cells in peripheral blood, their large number in lungs of asthmatic patients suggest their selective enrichment. It has been shown that iNK T cells can recognize the synthetic glycolipid α-galactosyl-ceramide, the self-glycolipid isoglobotrihexosyl-ceramide (iGb3), bacterial glycosphingolipids and glycolipids from plant pollens. A subset of non-iNK T cells also recognizes self-glycolipid sulfatide as well as other sulfatides and glycolipids of the present embodiments. One example of the mechanisms by which sulfatide controls autoimmunity involves the inactivation or non-responsiveness of iNK T cells, hence in some embodiments, sulfatide can be used to treat asthma in a patient.

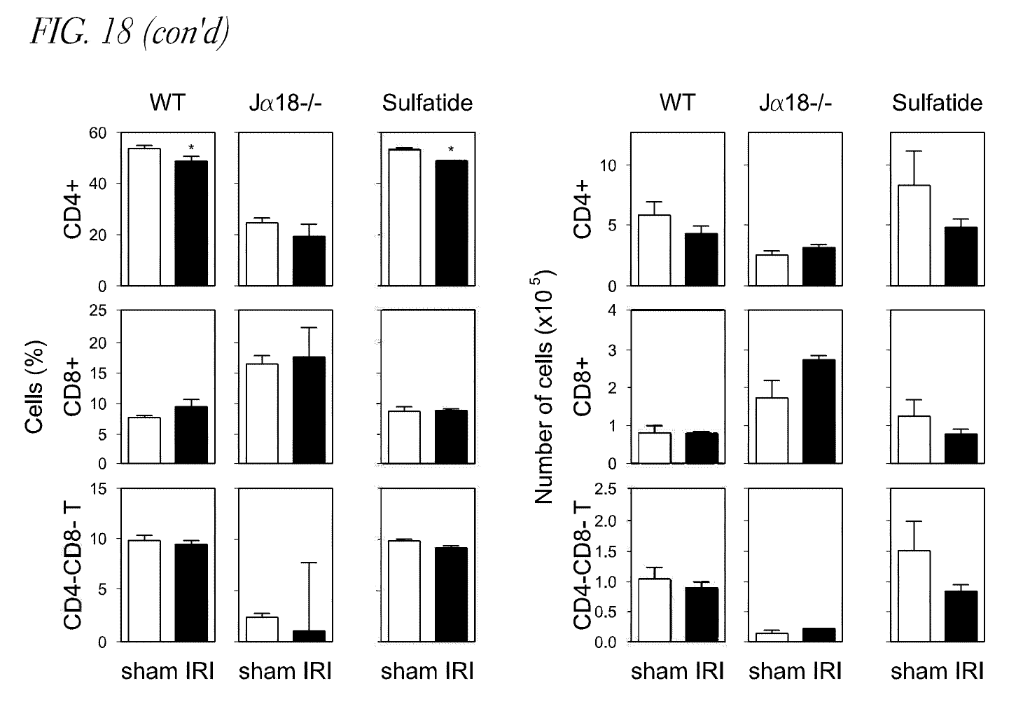

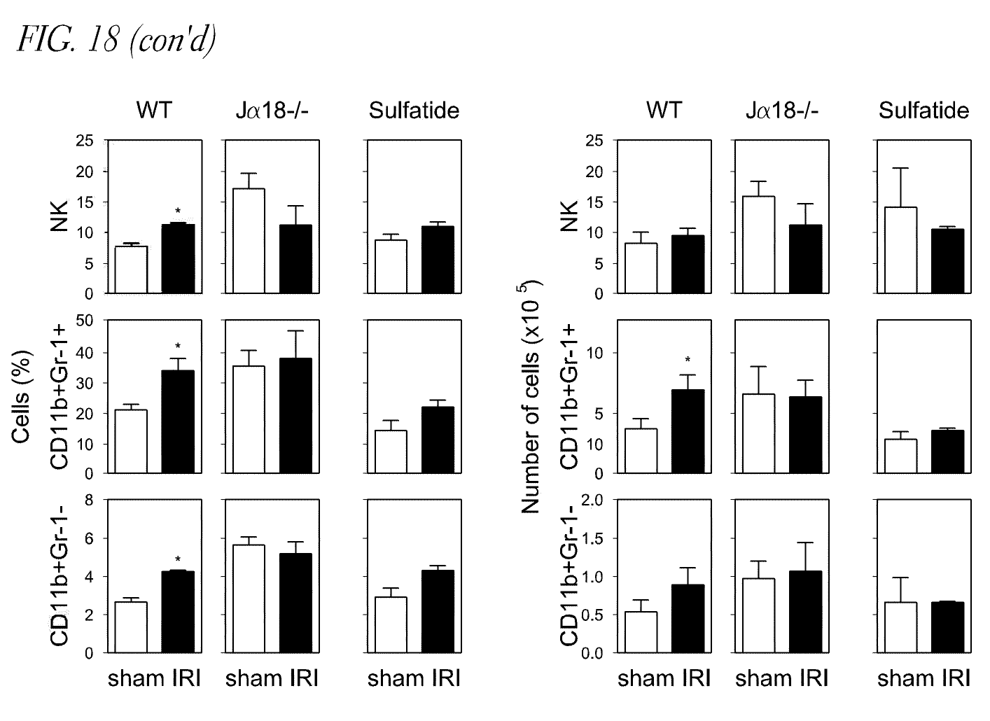

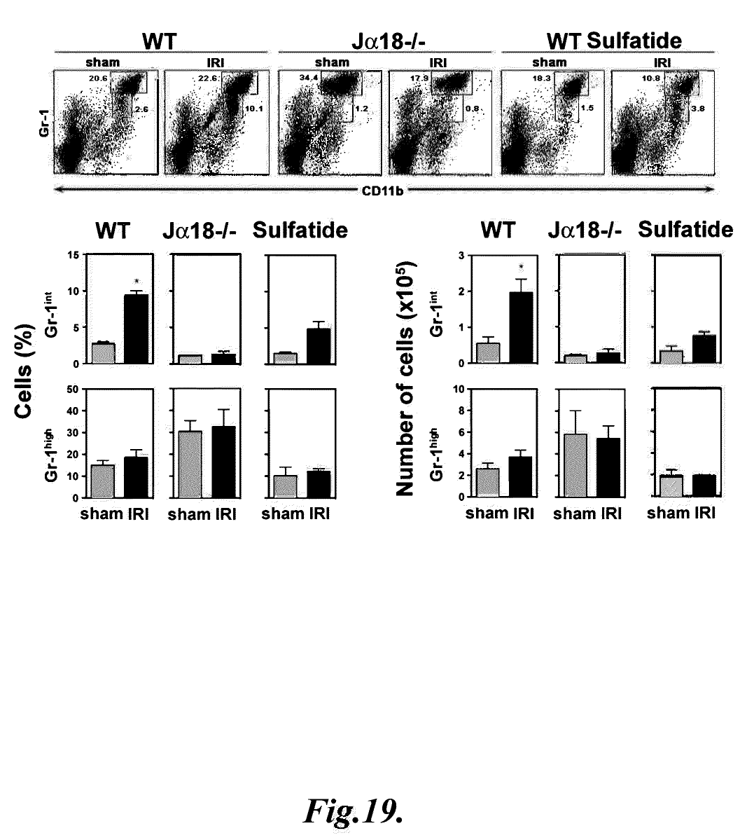

The development of ischemic reperfusion injury can be discussed in the context of at least two phases: an initial period (following from about 1 to about 6 hours of reperfusion) dominated by Kupffer cell activation, release of reactive oxygen species, CD4⁺ cell recruitment and secretion of proinflammatory cytokines. This initial phase is followed by a later period (following from about 6 to about 48 hours of reperfusion) characterized by neutrophil accumulation and induction of necrosis. Myeloid (CD11b⁺) cell subsets are recruited into reperfused tissues at in the early phase of ischemia and reperfusion injury. One example is the CD11b⁺Gr-1^{int} subset, comprising myeloid precursor cells and monocytes (see FIG. 18). Another example is the CD11b⁺Gr-1^{int} subset, containing macrophages and myeloid dendritic cells (see FIG. 19). The recruitment of myeloid cells other than neutrophils, such as the CD11b⁺Gr-1^{int} subset, suggests that injury is enhanced by recruitment of inflammatory monocytes into reperfused tissue.

Reperfusion injury can occur in a variety of tissues when blood supply is restored after a period of ischemia. Examples include skeletal muscle tissue following a crush injury, cardiac muscle in connection with a myocardial infarction or cardiac surgery, or ischemic heart disease, neural tissue in connection with a stroke or brain trauma, and hepatic and renal tissue in connection with surgery or trauma. Ischemic reperfusion injury also plays a major role in the quality and function of graft tissue in organ transplant. Ischemia and reperfusion injury is a major cause for increased length of hospitalization and decreased long-term graft survival.

In the murine model of hepatic ischemia and reperfusion injury, liver injury is significantly decreased in CD1d^{-/-} mice, which lack both type I and type II NKT cells, or in WT mice following treatment with blocking anti-CD1d antibodies. Further, serum ALT levels were also reduced following ischemia and reperfusion injury induction in CD1d^{-/-} mice lacking both type I and type II NKT cells. In both CD1d^{-/-} mice and mice treated with blocking anti-CD1d antibodies, levels of serum IFN-γ are reduced in ischemia and reperfusion injury compared to WT mice. Experiments with “blocking” anti-CD1d antibodies (Abs), however, may not be definitive, since anti-CD1d Abs act also as agonists in directly stimulating IL-12 production from Antigen-Presenting Cells (APCs).

As disclosed herein, type I NKT cells can have a pathogenic role in conditions such as ischemia and reperfusion injury. Some embodiments relate to a protective role for sulfatide-reactive type II NKT cells in injury following IR. Some embodiments relate to a protective role for sulfatide-reactive type II NKT cells in liver injury following IR. In the absence of type I NKT cells in Jα18^{-/-} mice or following the inactivation of type I NKT subsequent to sulfatide-mediated activation of type II NKT, liver tissue was significantly protected from hepatic ischemia and reperfusion injury, as less hepatocellular necrosis and reduced serum alanine aminotransferase (ALT) levels were observed. Furthermore, following activation of type II NKT cells with sulfatide, decreased IFN-γ secretion by hepatic type I NKT cells and prevention of liver injury were observed. Prevention of liver injury was associated with decreased recruitment of myeloid cell subsets and NK cells into the liver following activation of type II NKT cells and inhibition of type I NKT cells. Some embodiments disclosed herein relate to the use of sulfatide for treating or preventing tissue damage caused during liver transplantation and other surgeries associated with hepatic reperfusion.

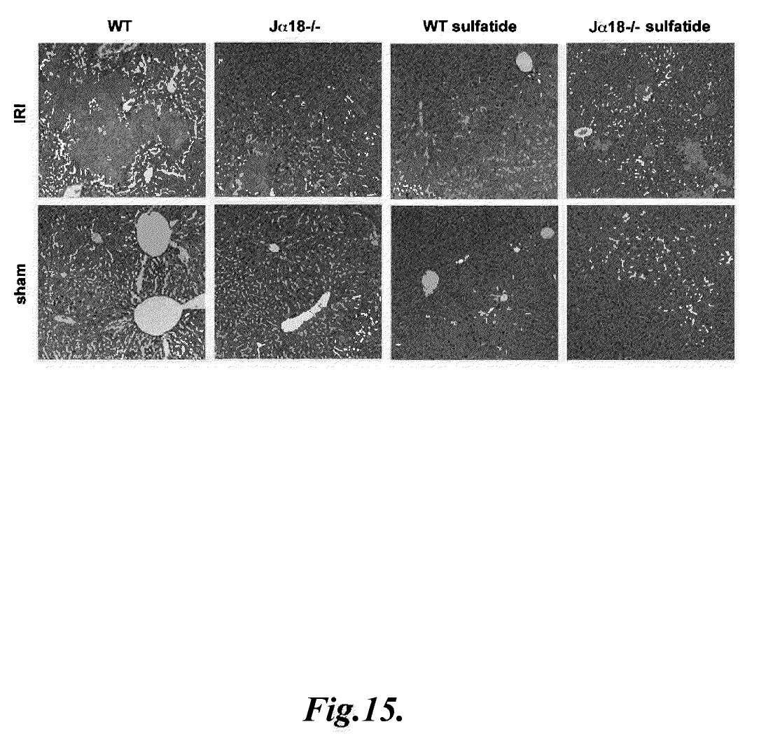

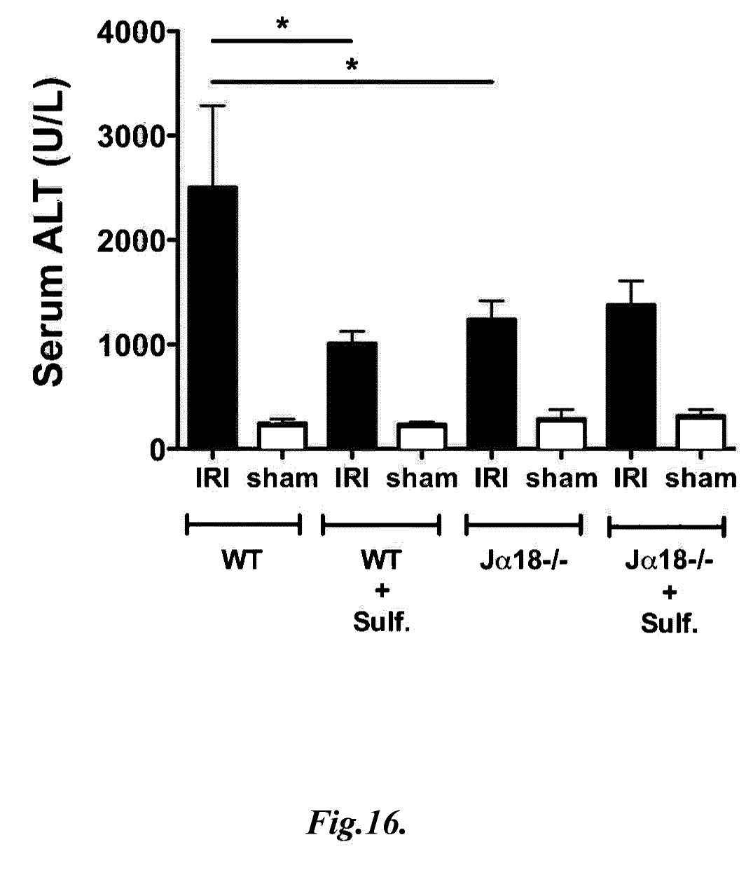

Liver injury following ischemia/reperfusion is significantly reduced in the absence of type I NKT cells, as assessed by histology and blood chemistry. The role of type I NKT cells in hepatic ischemia and reperfusion injury can be observed in Jα18^{-/-} mice, which lack type I NKT cells but have normal levels of type II NKT cells. As shown in FIG. 15, large necrotic areas were found in cephalad liver lobes of WT mice following 90 min of ischemia and 24 hrs of reperfusion, whereas necrotic areas in Jα18^{-/-} mice were remarkably reduced by comparison to WT mice. Further, serum alanine aminotransferase (ALT) enzyme levels, a marker of hepatocellular damage, were decreased in Jα18^{-/-} mice by ˜51% compared to WT mice (see FIG. 16). Serum ALT levels of CD1d^{-/-} mice lacking both type I and type II NKT cells were also reduced following ischemia and reperfusion injury induction. This indicates a pathogenic role for type I NKT cells in mediating hepatic ischemia and reperfusion injury.

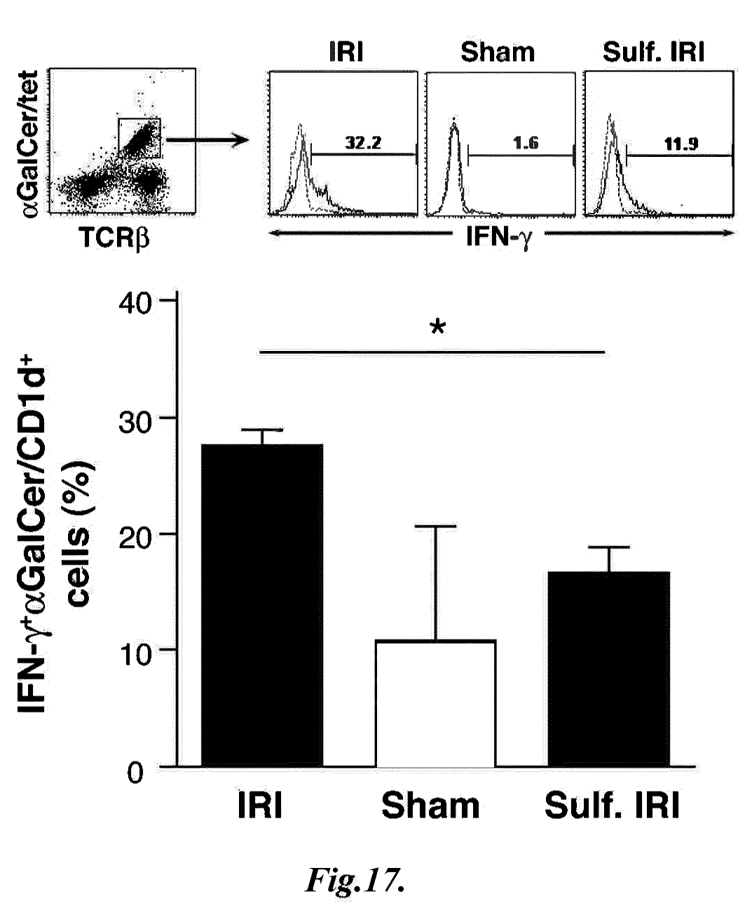

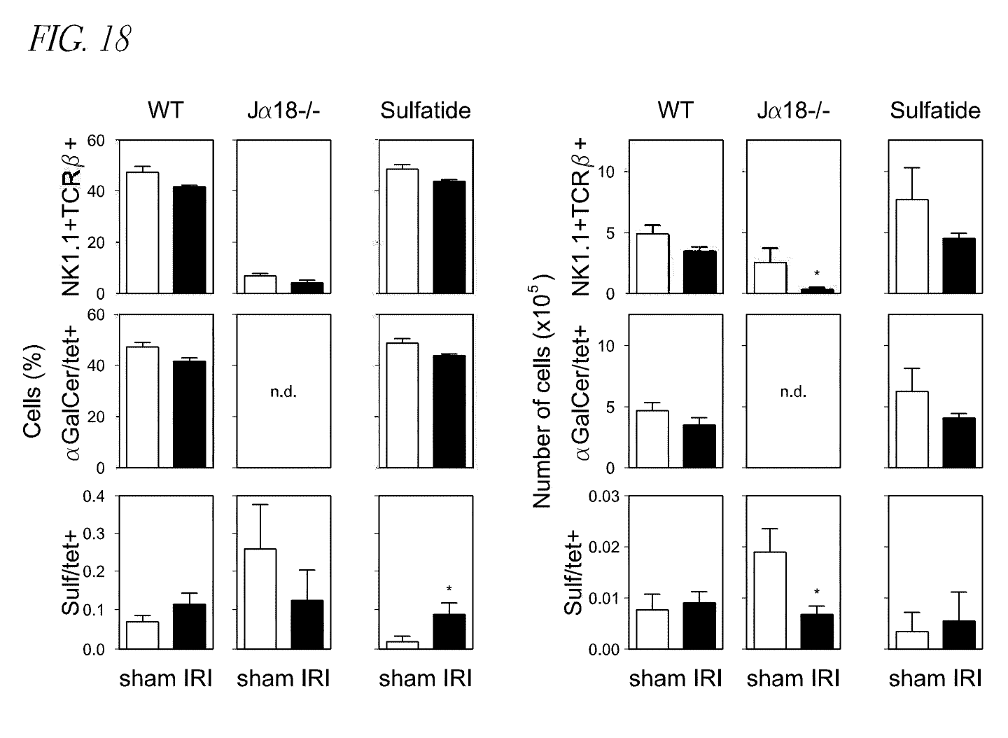

Increased intracellular IFN-γ expression by hepatic type I NKT cells during ischemia and reperfusion injury is associated with reperfusion injury. Hepatic type I NKT cells secrete IFN-γ during the early phase of ischemia and reperfusion injury (FIG. 17); however, secretion is significantly decreased following inactivation of type I NKT by sulfatide administration. This administration leads to protection of the liver from injury. The role of IFN-γ secretion by type I NKT cells may be a common feature of ischemic organ injury, as kidney ischemia and reperfusion injury is also attenuated in Jα18^{-/-} mice, which lack type 1 NKT.

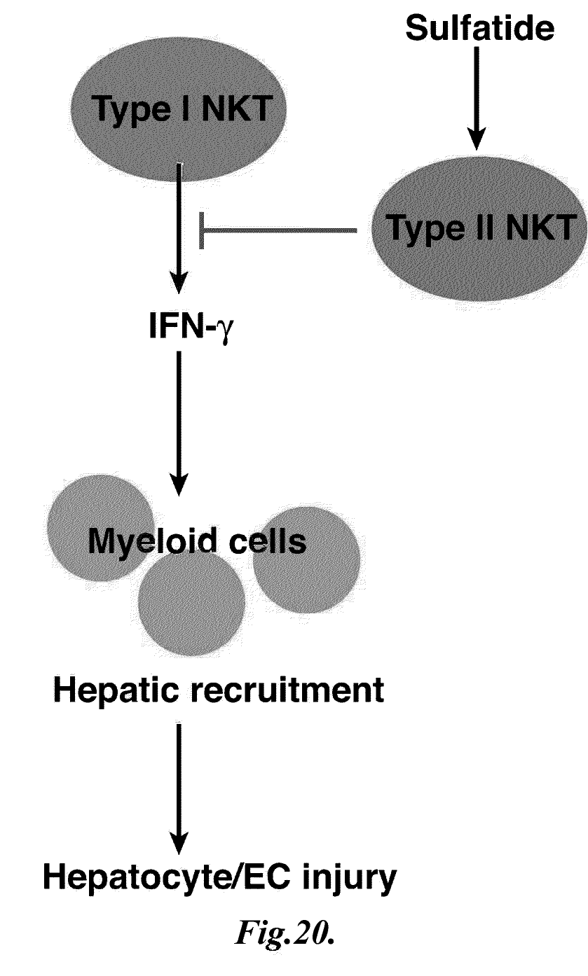

Several embodiments relate to a protective role in hepatic ischemia and reperfusion injury for a major subset of type II NKT cells, which are reactive to sulfatide. Activation of this subset by administration of sulfatide can prevent or treat many types of tissue injury. In some embodiments, intraperitoneal sulfatide administration from about 3 to about 48 hrs prior to ischemia induction effectively prevents liver injury. Sulfatide-mediated protection is associated with inhibition of IFN-γ secretion by hepatic type I NKT cells and suppression of type I NKT cell-mediated recruitment of myeloid cells, for example, the CD11b⁺Gr-1^{int} and Gr-1⁻ subsets, and NK cells into the liver. Without intending to be bound by a particular theory, a model of the activity of the different subsets of NKT cells in hepatic ischemia and reperfusion injury is summarized in FIG. 20.

Several embodiments relate to the inactivation of type I NKT cells and inhibition of IFN-γ secretion in reperfused livers by sulfatide administration. The extent of ischemia and reperfusion injury prevention following treatment with sulfatide is similar to that found in the total absence of type I NKT cells (Jα18^{-/-} mice). In both cases, ALT levels were reduced by >50%, and only minimal or no hepatic necrotic areas were present. This is consistent with a novel immunoregulatory pathway in which activation of type II NKT cells by sulfatide in vivo mediates anergy induction in type I NKT cells that is dependent upon IL-12 and modification of hepatic DC subsets. Adoptive transfer of hepatic CD11c⁺ cells from sulfatide-treated animals is sufficient to reduce liver damage in the recipients. Without intending to be bound by a particular theory, the protective effect of sulfatide pretreatment in hepatic ischemia and reperfusion injury likely results from inactivation of pathogenic type I NKT cells by CD11c⁺ myeloid cells modified through their interaction with sulfatide-activated type II NKT cells.

Several embodiments relate to the development of immunotherapeutics based upon the usage of sulfatide or its analogs to ameliorate tissue damage encountered during surgeries associated with hepatic reperfusion such as, for example, liver transplantation and hepatic resection. Such strategies would allow more patients to undergo successful liver transplantation by expanding the donor pool to those with organs that are currently highly susceptible to ischemia and reperfusion injury and by diminishing the level of overall immune activation predisposing to graft rejections.

Some embodiments relate to the activation of type II NKT cells by administration of sulfatide in vivo resulting in anergy induction or inactivation of type I NKT cells. This effect is associated with a significant inhibition in type I NKT cell proliferation and cytokine secretion in response to αGalCer.

Some embodiments relate to pretreatment of patients with a sulfatide prior to surgeries associated with hepatic reperfusion such as liver transplantation and hepatic resection. In several embodiments, patients are treated with an effective amount of sulfatide from about 1 second to about 24 hours prior to undergoing a surgical procedure. In some other embodiments, patients are treated with an effective amount of sulfatide from about 30 minutes to about 20 hours prior to undergoing a surgical procedure. In some other embodiments, patients are treated with an effective amount of sulfatide from about 1 hour to about 18 hours prior to undergoing a surgical procedure. In some other embodiments, patients are treated with an effective amount of sulfatide from about 2 hours to about 15 hours prior to undergoing a surgical procedure. In some other embodiments, patients are treated with an effective amount of sulfatide from about 4 hours to about 10 hours prior to undergoing a surgical procedure. In some other embodiments, patients are treated with an effective amount of sulfatide from about 3 hours to about 5 hours prior to undergoing a surgical procedure. In some embodiments, patients are treated with an effective amount of sulfatide about 1-3 hours, about 2-4, about 3-5 hours, about 4-6 hours, about 5-8 hours, about 7-10 hours, about 9-12 hours, about 11-15 hours, about 14-20 hours, or about 16-24 hours. In some embodiments, the treatment with an effective amount of sulfatide can be administered during surgery or after the surgery.

Sulfatide derived from bovine brain myelin is comprised of a 2:1 mixture of saturated/unsaturated major acyl chains (C₂₄), with unsaturation occurring at C₁₉. Several embodiments relate to the use of synthetic sulfatide analogs, such as analogs with saturated acyl chain (C₂₄) and unsaturated chains (C₂₄: 1) as well as analogs comprised of different lengths of acyl chains in the fatty acid or sphingosine moiety (shorter as well as longer, for example, C₁₈, C₃₂) and positional isomers with 3′ vs. 4′-sulfated group on the galactose moiety (3′-803 vs. 4′-803). Some embodiments relate to a synthetic product of an immunodominant species of sulfatide, cis-tetracosenoyl sulfatide, which also binds efficiently to the CD1d molecules and activates type II NKT cells.

Several embodiments relate to a method for testing the ability of synthetic sulfatides, such as cis-tetracosenoyl, and related glycolipid analogs, to activate type II NKT cells. Activation of type II NKT cells can be evaluated by assessing the in vitro proliferative response of type II NKT cells to synthetic sulfatide pulsed dendritic cells, synthetic-sulfatide-CD 1 d-tetramer staining, C069 expression and cytokine secretion profile of tetramer-positive cells by intracellular cytokine staining or real-time PCR for IFN-γ, IL-4 or IL-13. In addition, the ability of type II NKT cells activated with synthetic sulfatides, such as cis-tetracosenoyl, or related analogs, to anergize type I NKT cells can be evaluated by assessing the proliferative response of type I NKT cells to αGalCer (a potent activator of type I NKT cells) using CF8E-dilution analysis and intracellular cytokine staining of αGalCer/CO 1 d-tetramer cells. In some embodiments, a selected panel of synthetic sulfatides, such as cis-tetracosenoyl and related analogs that activate type II NKT cells will be evaluated for their ability to modulate NKT activity.

Some embodiments relate to a process for synthesizing selected sulfatide analogs. A novel process for synthesizing cis-tetracosenoyl and related analogs will be developed based on a previously reported process (Wu, Xing et al. 2005; Zajonc, Maricic et al. 2005) adapted to be compatible with cGMP manufacturing requirements.

As used herein, the term “patient” refers to the recipient of a therapeutic treatment and includes all organisms within the kingdom animalia. In preferred embodiments, the animal is within the family of mammals, such as humans, bovine, ovine, porcine, feline, buffalo, canine, goat, equine, donkey, deer and primates. The most preferred animal is human.

As used herein, the terms “treat” “treating” and “treatment” include “prevent” “preventing” and “prevention” respectively.

In some other embodiments, the sulfatide can be administered alone or in combination with another therapeutic compound. Any currently known therapeutic compound used in treatment of the target autoimmune disease or reperfusion injury can be used. In some embodiments, sulfatide can be administered in combination with hydrogen sulfide (H₂S). In some embodiments sulfatide can be administered in combination with antioxidants. In several embodiments, sulfatide can be administered in combination with inhibitors of Na⁺—H⁺ exchange. In one preferred embodiment, no adjuvant is used.

Many different modes and methods of administration of the sulfatide are contemplated. In some embodiments, delivery routes include, for example, intravenous, intraperitoneal, inhalation, intramuscular, subcutaneous, and oral administration or any other delivery route known in the art. Depending on the particular administration route, the dosage form may be, for example, solid, semisolid, liquid, vapor or aerosol preparation. The dosage form may include, for example, those additives, lubricants, stabilizers, buffers, coatings, and excipients as is standard in the art of pharmaceutical formulations

Many pharmaceutical formulations are contemplated. In some embodiments, the pharmaceutical formulations can be prepared by conventional methods using the following pharmaceutically acceptable vehicles or the like: excipients such as solvents (e.g., water, physiological saline), bulking agents and filling agents (e.g., lactose, starch, crystalline cellulose, mannitol, maltose, calcium hydrogenphosphate, soft silicic acid anhydride and calcium carbonate); auxiliaries such as solubilizing agents (e.g., ethanol and polysolvates), binding agents (e.g., starch, polyvinyl pyrrolidine, hydroxypropyl cellulose, ethylcellulose, carboxymethyl cellulose and gum arabic), disintegrating agents (e.g., starch and carboxymethyl cellulose calcium), lubricating agents (e.g., magnesium stearate, talc and hydrogenated oil), stabilizing agents (e.g., lactose, mannitol, maltose, polysolvates, macrogol, and polyoxyethylene hydrogenated castor oil), isotonic agents, wetting agents, lubricating agents, dispersing agents, buffering agents and solubilizing agents; and additives such as antioxidants, preservatives, flavoring and aromatizing agents, analgesic agents, stabilizing agents, coloring agents and sweetening agents.

If necessary, glycerol, dimethyacetamide, 70% sodium lactate, surfactants and alkaline substances (e.g., ethylenediamine, ethanol amine, sodium carbonate, arginine, meglumine and trisaminomethane) can also be added to various pharmaceutical formulations.

In the context of some embodiments, the dosage form can be that for oral administration. Oral dosage compositions for small intestinal delivery include, for example, solid capsules as well as liquid compositions which contain aqueous buffering agents that prevent the sulfatide or other ingredients from being significantly inactivated by gastric fluids in the stomach, thereby allowing the sulfatide to reach the small intestines. Examples of such aqueous buffering agents which can be employed in the present embodiments include, for example, bicarbonate buffer at a pH of from about 5.5 to about 8.7. Tablets can also be made gastroresistent by the addition of, e.g., cellulose acetate phthalate or cellulose acetate terephthalate.

In some embodiments, the specific amount of sulfatide administered to a patient will vary depending upon the disease or condition being treated, as well as the age, weight and sex of the patient being treated. Generally, to achieve such a final concentration in, e.g., the intestines or blood, the amount of sulfatide molecule in a single dosage composition of the present embodiments will generally be about 0.1 milligrams to about 100 milligrams, preferably about 2.0 milligrams to about 60 milligrams, more preferably about 20 milligrams to about 50 milligrams. Likewise, the amount of a secondary therapeutic compound in a single oral dosage composition of the present embodiments will generally be in the range of about 0.01 milligrams to about 1000 milligrams, more preferably about 0.1 milligrams to about 100 milligrams. Obviously, the exact dosage will vary with the disease or disorder being treated, the preferred ranges being readily determinable.

In another embodiment, the sulfatide can be combined with a pharmaceutically acceptable vehicle. Suitable pharmaceutically acceptable vehicles include, for example, phosphate buffered saline and PBS-tween. In one embodiment, 0.1-10 mg/kg body weight of sulfatide are administered to the patient. More preferably, 1-10 mg/kg body weight of sulfatide are administered. Preferably, this dosage is repeated each day as needed. Alternative dosages and dose schedules are discussed infra.

In the present embodiments, sulfatides can be administered to a patient suffering from autoimmune diseases to improve the patient's condition. Accordingly, patients suffering from one or more of the various indications of a autoimmune or immune related diseases and disorders such as multiple sclerosis, systemic lupus erythematsosus, AIDS, Alzheimer's disease, rheumatoid arthritis, insulin dependent diabetes mellitus, autoimmune hepatitis, asthma and celiac disease can be treated using sulfatides according to the present embodiments.

In some embodiments, sulfatides can be administered to a patient prior to undergoing surgery where reperfusion injury is a risk. In some embodiments sulfatides may be administered to a patient prior to undergoing cardiac surgery. Several embodiments relate to the administration of sulfatide to prevent or treat reperfusion injury associated with thrombolysis, percutaneous coronary intervention (PCI), coronary artery bypass grafting (CABG), and cardiac transplantation. In some embodiments sulfatide is administered to treat or prevent lung reperfusion injury. In some embodiments, sulfatides may be administered to a patient suffering from ischemic heart disease. In some embodiments sulfatides can be administered to a subject suffering from a stroke or other brain trauma. In several embodiments, sulfatides can be administered to a patient undergoing or at risk for myocardial infarction. In some embodiments, sulfatide can be administered to a patient after suffering a myocardial infarction. In several embodiments sulfatides may be administered to a patient suffering from a condition caused by repeated episodes of ischemia and reperfusion, such as pressure sores or diabetic foot ulcers. In some embodiments sulfatide can be administered to a patient having Sickle Cell Anemia. In some embodiments, sulfatides can be administered in connection with therapeutic hypothermia, which is used to limit ischemic injuries.

In accordance with the embodiments, sulfatides can be administered to alleviate a patient's symptoms, or can be administered to counteract a mechanism of the disorder itself. In certain embodiments, sulfatides may be administered as a prophalactic measure. In some embodiments multiple doses of sulfatide is administered. It will be appreciated by those of skill in the art that these treatment purposes are often related and that treatments can be tailored for particular patients based on various factors. These factors can include the age, gender, or health of the patient, and the progression of autoimmune or immune related disease or disorder. The treatment methodology for a patient can be tailored accordingly for dosage, timing of administration, route of administration, and by concurrent or sequential administration of other therapies.

Some embodiments relate to a kit which may include one or more sulfatides, preferably as a pharmaceutical composition. In some embodiments, cis-tetracosenoyl is provided in a pharmaceutically acceptable carrier. In several embodiments, hydrogen sulfide (H₂S) may optionally be provided. In several embodiments, kits may further comprise suitable packaging and/or instructions for use of the sulfatide. Kits may also comprise a means for the delivery of the sulfatide, such as an inhaler, spray dispenser (e.g., nasal spray), syringe for injection, needle, IV bag or pressure pack for capsules, tables, suppositories. The sulfatide can be in a dry or lyophilized form or in a solution, particularly a sterile solution. When the sulfatide is in a dry form, the kit may comprise a pharmaceutically acceptable diluent for preparing a liquid formulation. The kit may contain a device for administration or for dispensing the compositions, including, but not limited to, syringe, pipette, transdermal patch, or inhalant. Some embodiments relate to kits that contain sufficient dosages of the compounds or composition to provide effective treatment for an individual for an extended period, such as a week, 2 weeks, 3 weeks, 4 weeks, 6 weeks, or 8 weeks or more.

In one example embodiment, a 70 kg adult patient at risk of chemical liver damage from prescription drugs or drugs of abuse is given a daily i.m. injection of 70 mg sulfatide in 1.0 ml phosphate buffered saline to treat liver damage. This dosage can be adjusted based on the results of the treatment and the judgment of the attending physician. Treatment is preferably continued for at least about 1 or 2 weeks, preferably at least about 1 or 2 months, and may be continued on a chronic basis.

In another example embodiment, a 70 kg adult human is given a daily i.m. injection of 70 mg sulfatide in 1.0 ml phosphate buffered saline to treat AIDS. This dosage can be adjusted based on the results of the treatment and the judgment of the attending physician. Treatment is preferably continued for at least about 1 or 2 weeks, preferably at least about 1 or 2 months, and may be continued on a chronic basis.

In yet another example embodiment, and 70 kg adult human is given a daily i.m. injection of 70 mg sulfatide in 1.0 ml phosphate buffered saline to treat multiple sclerosis. This dosage can be adjusted based on the results of the treatment and the judgment of the attending physician. Treatment is preferably continued for at least about 1 or 2 weeks, preferably at least about 1 or 2 months, and may be continued on a chronic basis.

In still another example embodiment, a 70 kg adult human is given a daily i.m. injection of 70 mg sulfatide in 1.0 ml phosphate buffered saline to treat systemic lupus erythematsosus. This dosage can be adjusted based on the results of the treatment and the judgment of the attending physician. Treatment is preferably continued for at least about 1 or 2 weeks, preferably at least about 1 or 2 months, and may be continued on a chronic basis.

In another example embodiment, a 70 kg adult human is given a daily i.m. injection of 70 mg sulfatide in 1.0 ml phosphate buffered saline to treat Alzheimer's disease. This dosage can be adjusted based on the results of the treatment and the judgment of the attending physician. Treatment is preferably continued for at least about 1 or 2 weeks, preferably at least about 1 or 2 months, and may be continued on a chronic basis.

In another example embodiment, a 70 kg adult human is given a daily i.m. injection of 70 mg sulfatide in 1.0 ml phosphate buffered saline to treat rheumatoid arthritis. This dosage can be adjusted based on the results of the treatment and the judgment of the attending physician. Treatment is preferably continued for at least about 1 or 2 weeks, preferably at least about 1 or 2 months, and may be continued on a chronic basis.

In another example embodiment, a 70 kg adult human is given a daily i.m. injection of 70 mg sulfatide in 1.0 ml phosphate buffered saline to treat autoimmune hepatitis. This dosage can be adjusted based on the results of the treatment and the judgment of the attending physician. Treatment is preferably continued for at least about 1 or 2 weeks, preferably at least about 1 or 2 months, and may be continued on a chronic basis.

In another example embodiment, a 70 kg adult human is given a daily i.m. injection of 70 mg sulfatide in 1.0 ml phosphate buffered saline to treat celiac disease. This dosage can be adjusted based on the results of the treatment and the judgment of the attending physician. Treatment is preferably continued for at least about 1 or 2 weeks, preferably at least about 1 or 2 months, and may be continued on a chronic basis.

In another example embodiment, a 70 kg adult human is given a daily i.m. injection of 70 mg sulfatide in 1.0 ml phosphate buffered saline to treat insulin dependent diabetes mellitus. This dosage can be adjusted based on the results of the treatment and the judgment of the attending physician. Treatment is preferably continued for at least about 1 or 2 weeks, preferably at least about 1 or 2 months, and may be continued on a chronic basis.

In another example embodiment, a 70 kg adult human is given a daily i.m. injection of 70 mg sulfatide in 1.0 ml phosphate buffered saline to treat asthma. In the alternative, the 70 kg adult human is given a daily inhalation treatment of 70 mg sulfatide for the treatment of asthma. These dosages can be adjusted based on the results of the treatment and the judgment of the attending physician. Treatment is preferably continued for at least about 1 or 2 weeks, preferably at least about 1 or 2 months, and may be continued on a chronic basis.

In one example embodiment, a 70 kg adult patient is given an intraperitoneal injection of 70 mg sulfatide in 1.0 ml phosphate buffered saline 4 hours prior to hepatic resection surgery. This dosage can be adjusted based on the results of the treatment and the judgment of the attending physician. Additional doses may be administered just prior to or during surgery. Additional doses may be administered 4-6 hours after surgery and daily for about 1 or 2 weeks.

In one example embodiment, a 70 kg adult patient is given an intraperitoneal injection of 70 mg sulfatide in 1.0 ml phosphate buffered saline during hepatic resection surgery. This dosage can be adjusted based on the results of the treatment and the judgment of the attending physician. Additional doses may be administered 4-6 hours after surgery and daily for about 1 or 2 weeks.

In one example embodiment, a 70 kg adult patient is given an intraperitoneal injection of 70 mg sulfatide in 1.0 ml phosphate buffered saline 4-6 hours after hepatic resection surgery. This dosage can be adjusted based on the results of the treatment and the judgment of the attending physician. Additional doses may be administered and daily for about 1 or 2 weeks.

In another example embodiment, a 70 kg adult patient is given an intraperitoneal injection of 70 mg sulfatide in 1.0 ml phosphate buffered saline upon presenting with symptoms of myocardial infarction. This dosage can be adjusted based on the results of the treatment and the judgment of the attending physician. Additional doses may be administered daily for about 1 or 2 weeks.

In one example embodiment, a 70 kg adult patient is given 70 mg sulfatide dissolved in 1.0 ml phosphate buffered saline added to an I.V. drip 4 hours prior to cardiac surgery. This dosage can be adjusted based on the results of the treatment and the judgment of the attending physician. Additional doses may be administered just prior to or during surgery. Additional doses may be administered 4-6 hours after surgery and daily for about 1 or 2 weeks.

In another example embodiment, a 70 kg adult patient is given an intraperitoneal injection of 70 mg sulfatide in 1.0 ml phosphate buffered saline upon presenting with symptoms of a stroke. This dosage can be adjusted based on the results of the treatment and the judgment of the attending physician. Additional doses may be administered daily for about 1 or 2 weeks.

In one example embodiment, a 70 kg adult organ donor is given an intraperitoneal injection of 70 mg sulfatide in 1.0 ml phosphate buffered saline 4 hours prior to organ donation. This dosage can be adjusted based on the results of the treatment and the judgment of the attending physician. Additional doses may be administered just prior to or during surgery to harvest the organ(s).

In another example embodiment, a 70 kg adult patient is given an intraperitoneal injection of 70 mg sulfatide in 1.0 ml phosphate buffered saline 4 hours prior to receiving an organ transplant. This dosage can be adjusted based on the results of the treatment and the judgment of the attending physician. Additional doses may be administered just prior to or during surgery. Additional doses may be administered 4-6 hours after surgery and daily for about 1 or 2 weeks.

In another example embodiment, a 70 kg adult human having sickle cell anemia is given a daily i.m. injection of 70 mg sulfatide in 1.0 ml phosphate buffered saline to tissue damage resulting from poor circulation. These dosages can be adjusted based on the results of the treatment and the judgment of the attending physician. Treatment is preferably continued for at least about 1 or 2 weeks, preferably at least about 1 or 2 months, and may be continued on a chronic basis.

The following examples are provided for illustrative purposes only, and are in no way intended to limit the scope of the present embodiments.

### Example 1

### Sulfatide Treatment of Chronic Experimental Autoimmune Encephalomyelitis (EAE)

### C57.BL/6J Mouse Study

Wild type, C57.BL/6J female mice, 6-8 week of age were immunized once subcutaneously with 200 μg of myelin oligodendrocyte glycoprotein peptide MOG35-55 emulsified in incomplete Freund's adjuvant (DIFCO) supplemented with attenuated M. tuberculosis (DIFCO) to 1.65 mg/ml. 0.15 μg of pertussis toxin (PTx; List Biological Laboratories, Inc.) was injected twice in 200 μA saline intraperitoneally 0 and 48 h later. Mice were observed daily for signs of EAE for 40 days. The average disease score for each group was calculated by averaging the maximum severity of all of the affected animals in the group. Disease severity was scored on a 5-point scale, as described earlier: 1, flaccid tail; 2, hind limb weakness; 3, hind limb paralysis; 4, whole body paralysis; 5, moribund or death.

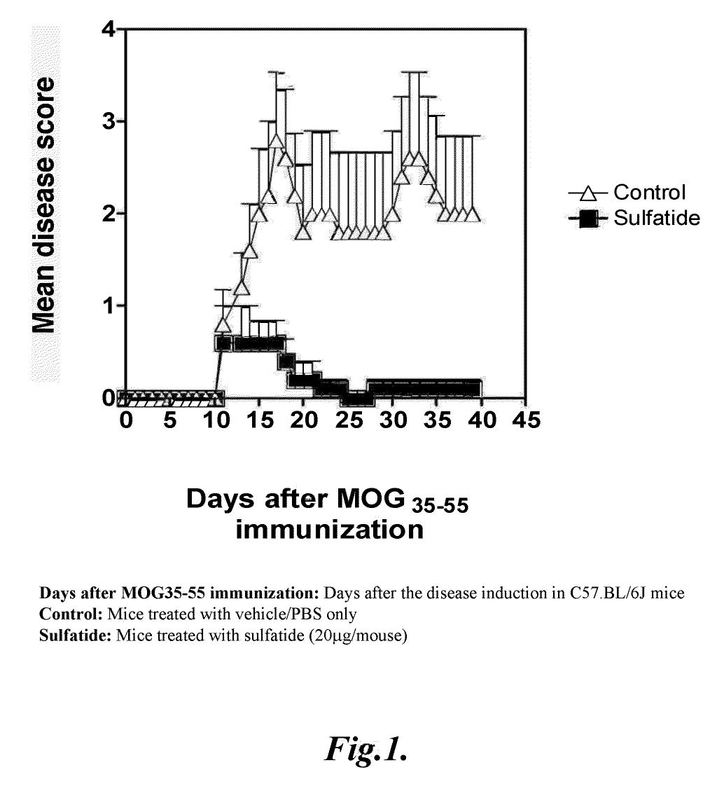

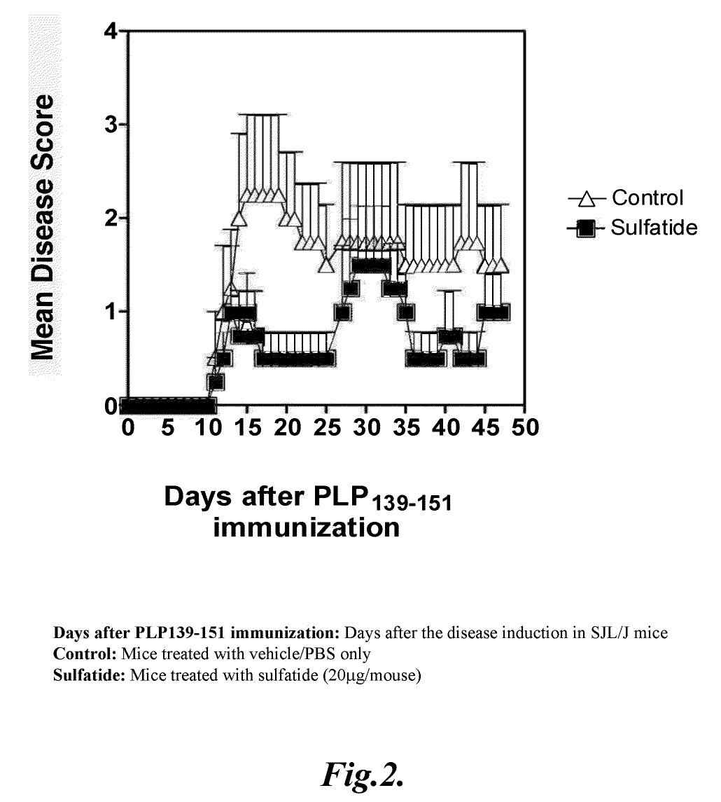

In the treatment protocol, 20 μg of bovine brain sulfatide in 200 μl of PBS or vehicle was given intraperitoneally (either three times, 1 wk apart, or once as indicated) at the onset of EAE. In the prevention protocol, 20 μg of sulfatide dissolved in 200 μl PBS was given intraperitoneally at the time of EAE induction. Results shown in FIG. 1 demonstrate that treatment with sulfatide reverses ongoing chronic EAE in C57.BL/6J mice.

### Example 2

### Sulfatide Treatment of Chronic and Relapsing Experimental Autoimmune Encephalomyelitis

### SJL/J Mouse Study

Wild type, SJL/J female mice, 6-8 week of age were immunized once subcutaneously with 75 μg of proteolipid protein peptide PLP139-151 emulsified in incomplete Freund's adjuvant (Difco, Detroit, Mich., USA) supplemented with attenuated M. tuberculosis (DIFCO) to 2 mg/ml. Mice were observed daily for signs of EAE for 50 days. The average disease score for each group was calculated by averaging the maximum severity of all of the affected animals in the group. Disease severity was scored on a 5-point scale, as described earlier: 1, flaccid tail; 2, hind limb weakness; 3, hind limb paralysis; 4, whole body paralysis; 5, moribund or death.

In the treatment protocol, 20 μg of bovine brain sulfatide in 200 μl of PBS or vehicle was given intraperitoneally at the onset of EAE and 2 weeks later. In the prevention protocol, 20 μg of sulfatide dissolved in 200 μl PBS was given intraperitoneally at the time of EAE induction. Results shown in FIG. 2 demonstrate that treatment of mice with sulfatide reverses ongoing chronic-relapsing EAE in SJL/J mice.

### Example 3

### Cis-tetracosenoyl Sulfatide Treatment of Chronic and Relapsing Experimental Autoimmune Encephalomyelitis (EAE)

### SJL/J Mouse Study

Wild type, SJL/J female mice, 6-8 wk of age were immunized once subcutaneously with 75 μg of PLP139-151 peptide emulsified in IFA (DIFCO) supplemented with attenuated M. tuberculosis (DIFCO) to 2 mg/ml. Mice were observed daily for signs of EAE for 50 days. The average disease score for each group was calculated by averaging the maximum severity of all of the affected animals in the group. Disease severity was scored on a 5-point scale, as described earlier: 1, flaccid tail; 2, hind limb weakness; 3, hind limb paralysis; 4, whole body paralysis; 5, moribund or death.

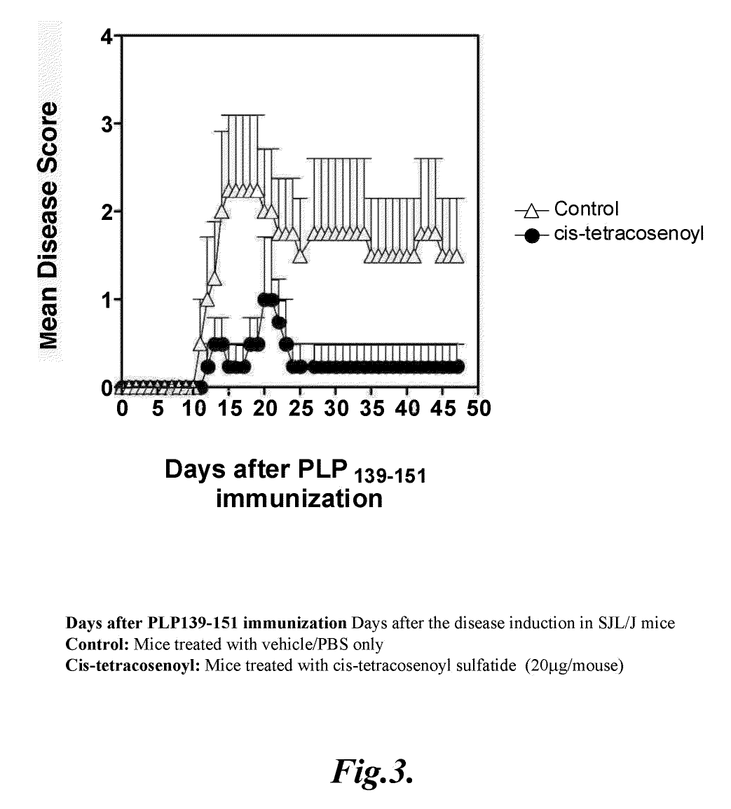

In the treatment protocol, 20 μg of semi-synthetic, cis-tetracosenoyl sulfatide (Formula (III)) in 200 μl of PBS or vehicle was given intraperitoneally at the onset of EAE. Results shown in FIG. 3 demonstrate that treatment of mice with cis-teracosenoyl sulfatide reverses ongoing chronic-relapsing EAE in SJL/J mice

### Example 4

### Sulfatide Treatment of Diabetes

### Non-Obese Diabetic or NOD Mouse Study

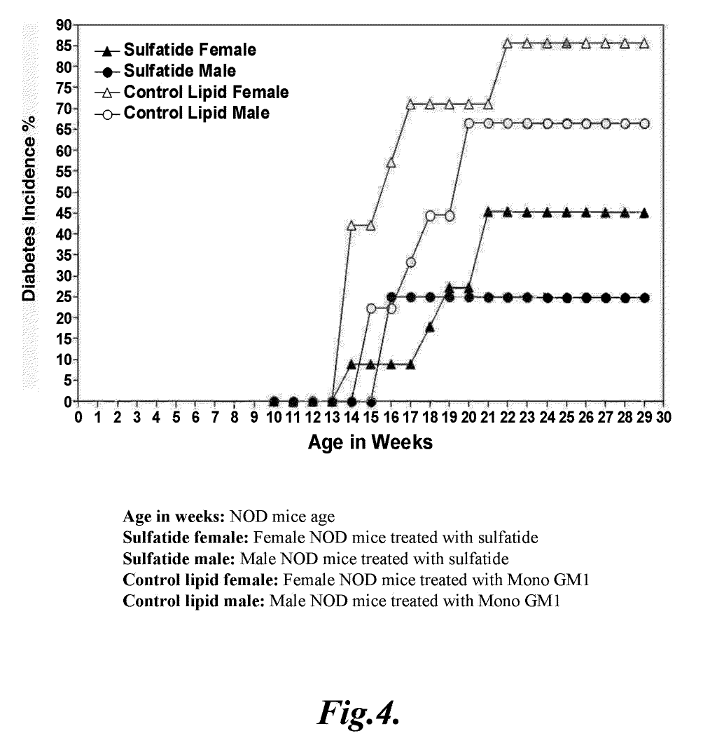

Groups of age-matched, 3 weeks old NOD mice (10-11 in each group) were given 3 weekly intraperitoneal injections of 20 μg bovine brain sulfatide or control lipid (Mono GM-1) in vehicle/PBS. Mice were monitored weekly for the glucose levels in urine and blood. The diabetes was diagnosed when blood glucose levels were >250 mg/dL in two consecutive readings. These data are representative of two independent experiments. Results shown in FIG. 4 demonstrate that administration of sulfatide in NOD mice prevents diabetes.

### Example 5

### Liver Injury Study Following Sulfatide Treatment of Autoimmune Hepatitis

Con A model: A dose of 8.5 mg/kg of Concanavalin A (Con A) (dissolved in pyrogen free phosphate buffer saline, PBS) was injected into female C57BL/6 mice intravenously (i.v.). 20 μg (1 mg/kg/m) of bovine brain sulfatide was administered intraperitonially (i.p.) immediately after Con A injection. Control mice were injected in parallel with PBS.

The serum was collected following administration with Con A or Con A+sulfatide and kept at −20° C. until use. The serum enzymes were measured at 0, 6, 12, 24, 48 and 72 hours following Con A or Con A+sulfatide injection. Serum levels ALT and AST were determined with the help of Laboratory Corporation of America, San Diego, Calif.

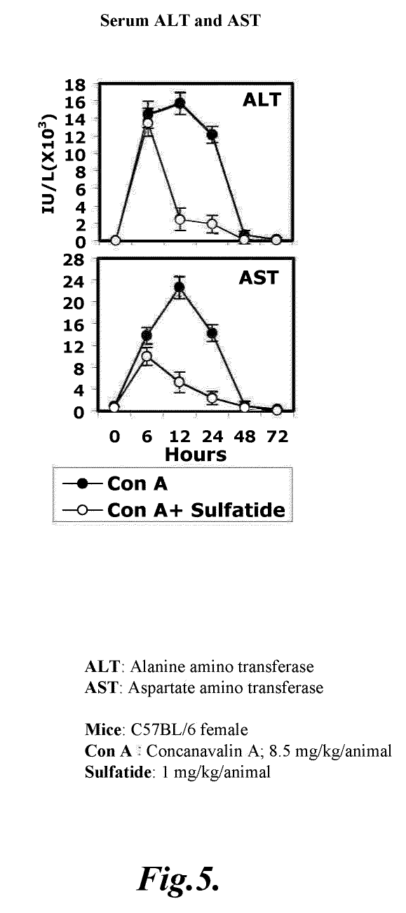

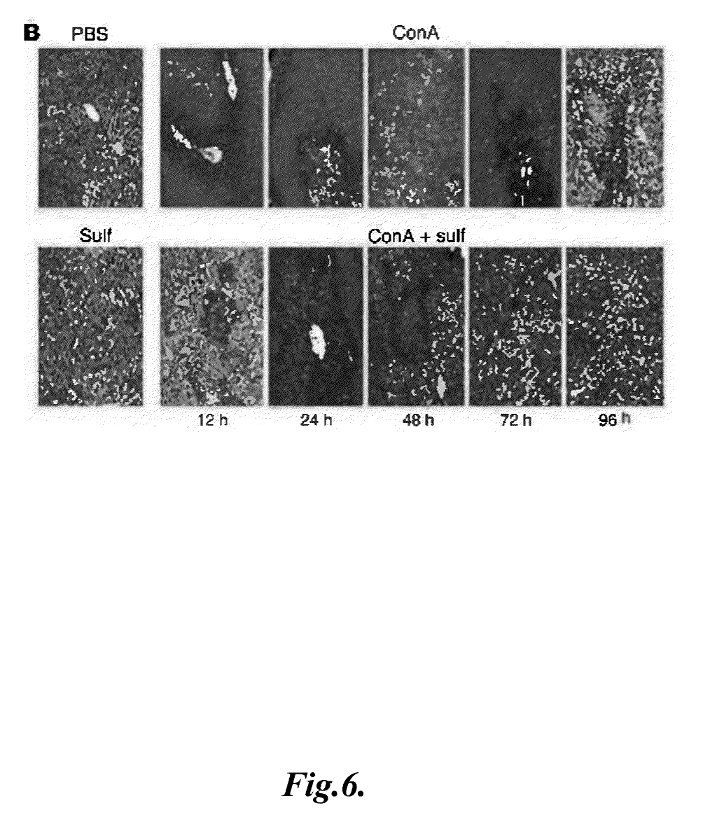

Comparable levels of serum enzymes, alanine amino transferase and aspartate amino transferase were observed 6 hours after Con A or Con A+sulfatide injected mice. Con A () injected mice, serum ALT and AST peaked around 12 h (ALT≅15.8×10³ IU/L and AST≅22.7×10³ IU/L) and returned to base line by 48 hours. In contrast, following combination of Con A+sulfatide (◯) injection (FIG. 5), a significant decrease in serum level of ALT and AST (ALT≅2.5×10³ IU/L and AST≅5.4×10³ IU/L) by 12 h was recorded and returned to base line by 24 h. p<0.0001 at 12 h.

### Example 6

### A Dramatic Improvement in Hepatitis Induced Liver Tissue Damage in Mice Treated with Sulfatide

Liver tissue was fixed in 10% formaldehyde solution at the indicated time points and kept at room temperature until use. Histological examination using hematoxylin and eosin staining was performed at Pacific Pathology Inc., San Diego, Calif.

A representative H&E-stained liver sections demonstrating markedly improved hepatic histology in Con A+bovine brain sulfatide treatment mice relative to Con A treatment mice in the indicated time points. Histological examination showed diffuse and massive infiltration and severe necrosis at the indicated time points following Con A injection mice, top panel. In contrast, sulfatide+Con A injection was associated with mild injury in terms of less infiltration and less necrosis in the 12 h to 48 h liver sections and histology returned to normal by 72 h, bottom panel. The bottom panel, left corner liver section represented at 24 h following only sulfatide injection was a control.

### Example 7

### Protection from Liver Damage from Hepatitis in Mice Injected with Sulfatide

Liver gross morphology was examined on days 3, 4 and 7 following Con A or Con A+Sulfatide injected mice. Representative liver photographs demonstrating severe necrosis (white spot) in only Con A treated mice (middle panel) but not in Con A+bovine brain sulfatide treated mice (lower panel) at the indicated times. Top panel shows PBS or sulfatide alone following 24 hours injection.

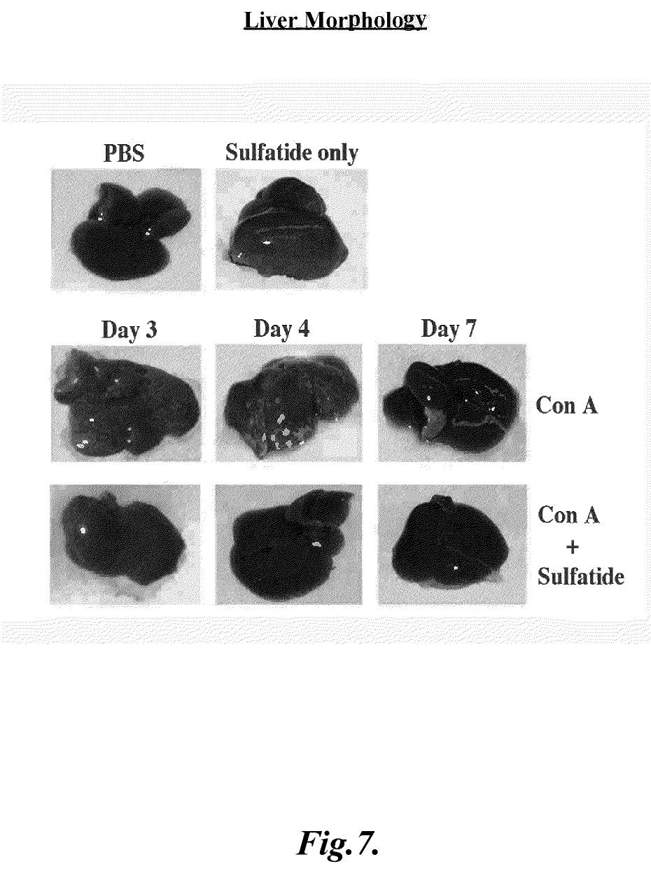

As shown in FIG. 7, a severe necrosis (white spots) was observed by macroscopic view of whole liver photographs on days 3, 4, and 7 following Con A-induced hepatitis mice. There was no liver necrosis (white spots) on days 3, 4, and 7 following a combination of Con A+sulfatide injection (bottom panel) compared to the PBS or sulfatide (24 h) injection (top panel). These results show that co-injection with sulfatide protects against Con A-induced hepatitis.

### Example 8

### Sulfatide Treatment Prevents HIV-1 Infection and Replication in SCID-hu Mice

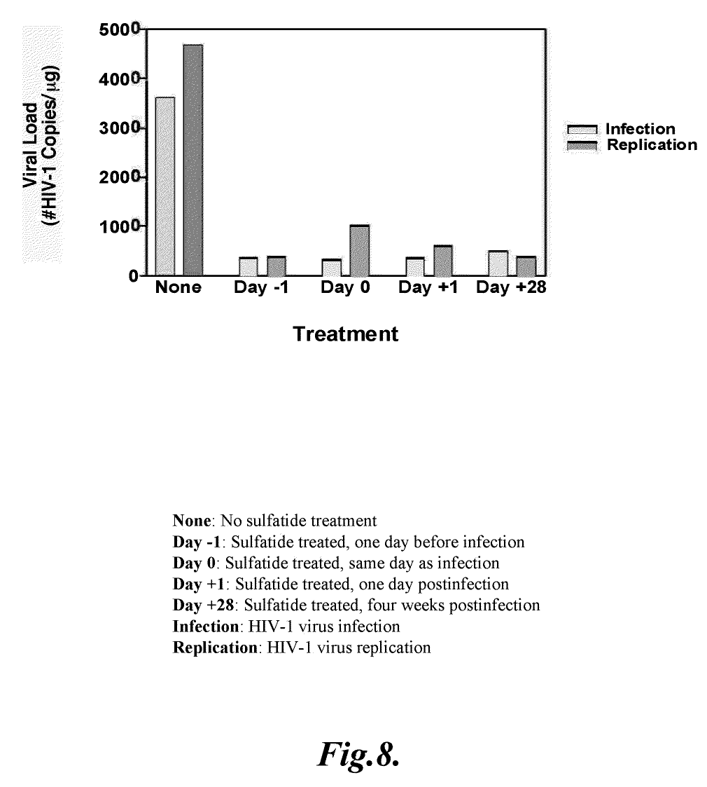

Thymus/liver implants of SCID-hu mice were infected with HIV-1 virus. Mice were given 20 μg of bovine brain sulfatide (Formula I) in 200 μl of PBS, intraperitoneally, twice a week at various points of viral infection of the animals. Thymus/liver implants from these mice were extracted and HIV-1 viral load was determined, including viral infection and replication. Results shown in FIG. 8 demonstrate that administration of sulfatide in SCID-hu mice drastically lowers the HIV-1 infection as well as the HIV-1 replication.

### Example 9

### Sulfatide Treatment Restores the Thymopoetic Potential of Human Implants in SCID-hu Mice

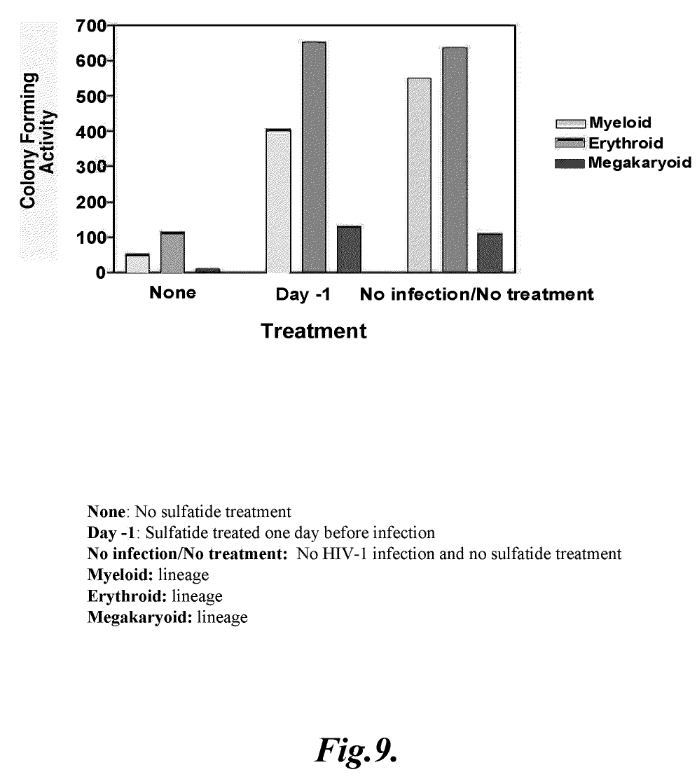

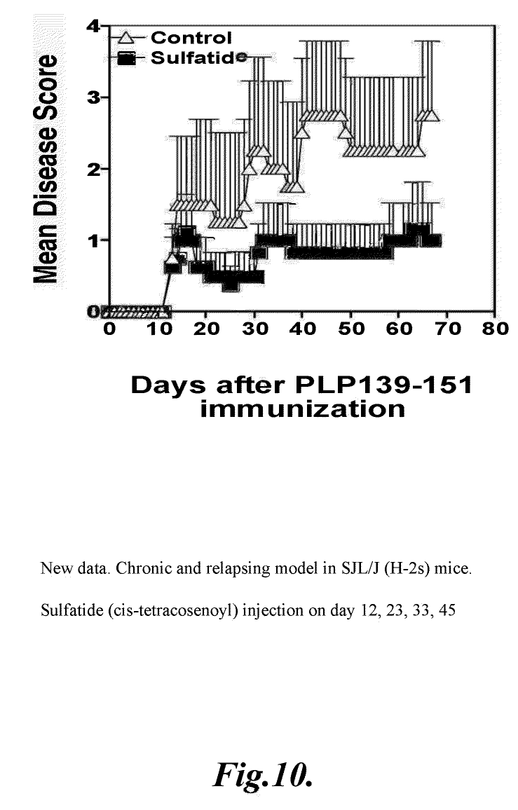

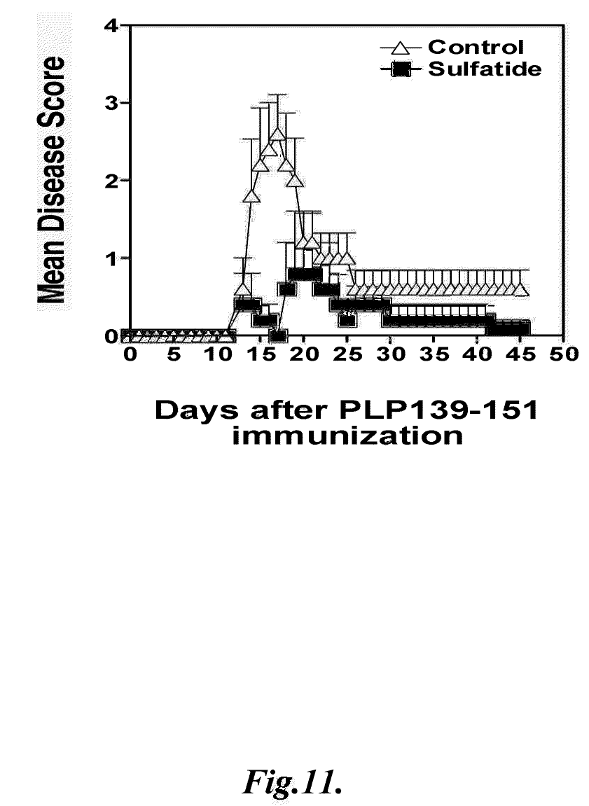

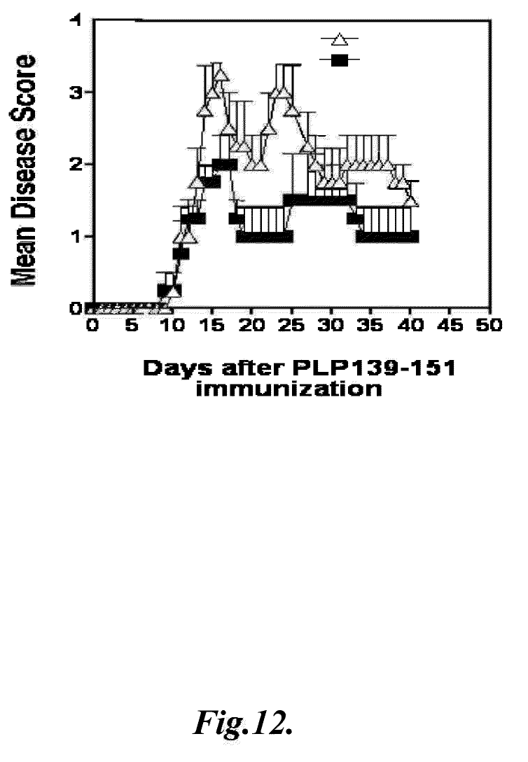

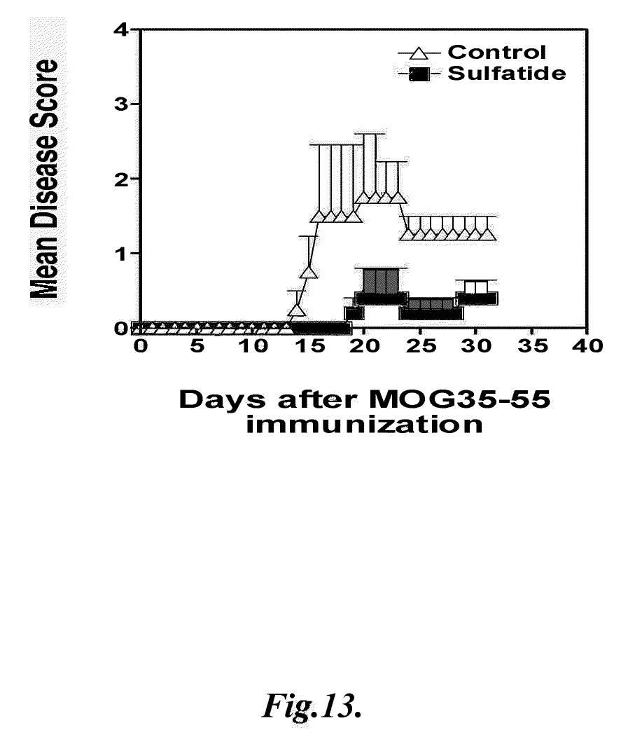

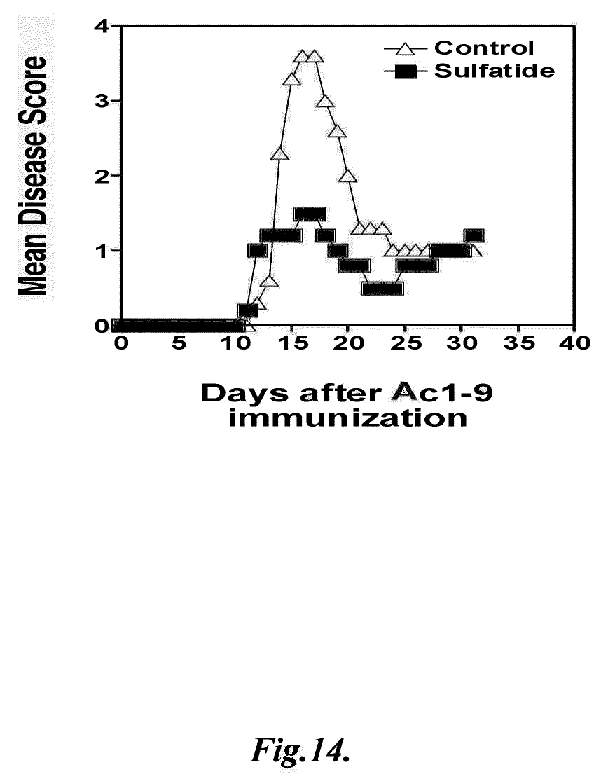

Thymus/liver implants of SCID-hu mice were infected with HIV-1 virus. Mice were given 20 μg of bovine brain sulfatide in 200 μl of PBS, intraperitoneally, twice a week at various time points in relation to the viral infection of animals. Multilineage hematopoiesis was assessed in vitro by colony forming activity of 5×10⁶ total cells derived from Thy/Liv implants, in methylcellulose (myeloid and erythroid), and in megacult-C (megakaryoid) membranes. Results shown in FIG. 9 demonstrate that administration of sulfatide in SCID-hu mice maintains the multilineage colony forming activity of human thymocytes during HIV-1 infection.

### Example 10

### Hepatic Ischemia and Reperfusion Injury Model

The hepatic ischemia and reperfusion injury model was established as described in Shen X D, Ke B, Zhai Y, et al., CD154-CD40 T-cell costimulation pathway is required in the mechanism of hepatic ischemia/reperfusion injury, and its blockade facilitates and depends on heme oxygenase-1 mediated cytoprotection. Transplantation 2002, 74:315-9, incorporated herein in its entirety, with few modifications. Female mice (8-16 weeks, age-matched) were anesthetized by intraperitoneal injection of 60 mg/kg sodium pentobarbital. After midline laparotomy, an atraumatic clip was applied to the hepatic triad (hepatic artery, portal vein, bile duct) of the 3 cephalad liver lobes. The caudal lobes retained intact blood circulation to prevent intestinal venous congestion. The peritoneum was closed and mice were placed on a heating pad (˜37° C.). Ambient temperature ranged between 25-26° C. After 90 min of partial hepatic warm ischemia, the clip was removed, initiating reperfusion, and the abdominal wall was sutured. Mice were euthanized after 6 or 24 hours of reperfusion, and blood and cephalad liver lobes were collected. Sham controls underwent the same procedure but without vascular occlusion.

### Example 11

### Hepatic Necroses are Reduced in the Absence of Type I NKT Cell

The extent of liver injury was analyzed in groups of WT and Jα18^{-/-} mice, which lack type I NKT cells but have normal levels of type II NKT cells. Hepatic ischemia and reperfusion injury was induced as described in Example 10 so that the WT and Jα18^{-/-} mice were subjected to 90 min of hepatic ischemia followed by 24 hrs of reperfusion or sham surgery. Liver tissues (cephalad lobes) were fixed in 10% formalin, embedded in paraffin and sections were stained with hematotoxylin and eosin for histological analysis (IDEXX Laboratories Inc., Westbrook, Me.). As shown in FIG. 15, large necrotic areas were found in cephalad liver lobes of WT mice following 90 min of ischemia and 24 hrs of reperfusion, whereas necrotic areas in Jα18^{-/-} mice were remarkably reduced. WT and Jα18^{-/-} mice subjected to sham surgery did not show any necrotic areas.

### Example 12

### Hepatic Necroses are Reduced Following Activation of Type II NKT Cells by Administration of Sulfatide

Purified bovine myelin-derived sulfatide (>90% pure), purchased from Matreya Inc., Pleasant Gap, Pa., was dissolved in vehicle (0.5% polysorbate-20 (Tween-20) and 0.9% NaCl solution) and diluted in PBS. Groups of BL/6 mice (WT sulfatide) and Jα18^{-/-} mice (Jα18 sulfatide) were treated with sulfatide (20 μg/mouse) intraperitoneally 3 to 48 hrs prior to ischemia induction or sham surgery. Hepatic ischemia and reperfusion injury and sham surgery were conducted as described in Example 10. Following 90 min of ischemia and 24 hrs of reperfusion, liver tissues (cephalad lobes) were fixed in 10% formalin, embedded in paraffin and sections were stained with hematotoxylin and eosin for histological analysis (IDEXX Laboratories Inc., Westbrook, Me.). As shown in FIG. 15, WT mice pretreated with sulfatide (WT sulfatide) 3 hrs prior to ischemia induction followed by 24 hrs of reperfusion developed only minimal or no hepatic necrosis, whereas large necrotic areas were found in the cephalad liver lobes of the untreated (WT) mice. Sham controls pretreated with sulfatide showed no necrosis.

### Example 13

### Levels of Serum ALT Induced in Response to Ischemia and Reperfusion Injury are Reduced in the Absence of Type I NKT Cells or Following Activation of Type II NKT Cells

Alanine aminotransferase (ALT) enzyme levels, a marker of heptocelluar damage, were assessed in WT and Jα18^{-/-} mice subjected to ischemia and reperfusion injury or sham surgery. Hepatic ischemia and reperfusion injury and sham surgery were performed as described in Example 10 on groups (1-2 mice/group) of WT and Jα18^{-/-} mice. Following 6 hrs of reperfusion or sham surgeries, blood was then obtained by cardiac puncture and serum was isolated. Serum Alanine aminotransferase (ALT) levels were determined on Olympus 5400 chemistry analyzer (IDEXX Laboratories Inc., Westbrook, Me.). In Jα18^{-/-} mice, serum ALT levels were decreased by ˜51% compared to WT mice (1238.1±178.4 U/I vs. 2502.0±783.2 U/I, P<0.05) (see FIG. 16). Among sham controls serum ALT levels were quite similar, regardless of presence of type I NKT cells, and means ranged between 204.0-285.1 U/I.

### Example 14

### Levels of Serum ALT Induced in Response to Ischemia and Reperfusion Injury are Reduced Following Activation of Type II NKT Cells

Alanine aminotransferase (ALT) enzyme levels, a marker of heptocelluar damage, were assessed in mice treated with sulfatide and subjected to ischemia and reperfusion injury or sham surgery. Sulfatide was obtained and prepared as described in Example 12. Hepatic ischemia and reperfusion injury and sham surgery were performed as described in Example 10 on groups (1-2 mice/group) of WT and Jα18^{-/-} mice treated with 20 ng sulfatide/mouse i.p. at 3, 16 or 48 hrs prior to surgery. In parallel, ischemia and reperfusion injury induction or sham surgeries were performed on WT and Jα18^{-/-} mice injected with vehicle. Following 6 hrs of reperfusion or sham surgeries, blood was obtained by cardiac puncture and serum was isolated. Serum Alanine aminotransferase (ALT) levels were determined on Olympus 5400 chemistry analyzer (IDEXX Laboratories Inc., Westbrook, Me.).

No differences in ALT levels were seen between mice injected with sulfatide at the different time points (3, 16 or 48 hrs prior to surgery), so the groups were pooled in FIG. 16. Vehicle-treated mice and untreated mice did not differ in the ALT levels (data not shown). As shown in FIG. 16, WT mice pretreated with sulfatide showed a significant decrease (˜60%) in serum ALT levels compared to untreated WT mice (1008.0±117.8 U/I vs. 2502.0±783.2 U/I, p<0.05). Sulfatide pretreatment of Jα18^{-/-} mice did not further reduce the ALT-levels in injured mice (FIG. 16). These data indicate that sulfatide mediated inactivation of type I NKT cells accounts for the protection from hepatic ischemia and reperfusion injury.

### Example 15

### Sulfatide Administration Prior to Ischemia and Reperfusion Injury Induction Significantly Inhibits IFN-γ Secretion by Type I NKT Cells

Sulfatide was obtained and prepared as described in Example 12. One group of WT mice (n=3) was injected with sulfatide (20 μg/mouse i.p.) 3 hrs prior to ischemia induction (Sulf. IRI), the other groups (IRI (n=3), sham (n=2)) were not pretreated. Hepatic ischemia and reperfusion injury (90 min of ischemia and 6 hrs of reperfusion) and sham surgery were performed as described in Example 10.

Cell preparation. Leukocytes were isolated from murine cephalad liver lobes, using mechanical crushing followed by Percoll gradient separation and RBC lysis as described in Halder R C, Aguilera C, Maricic I, et al., Type II NKT cell-mediated anergy induction in type I NKT prevents inflammatory liver disease. J Clin Invest 2007, 117:2302-12.

Flow cytometry. Leukocytes were suspended in FACS buffer (PBS containing 0.02% NaN₃ and 2% FCS), blocked (anti-mouse FcR-γ, BD Pharmingen, San Diego, Calif.) and stained with αGalCer loaded mCD1d-tetramer-PE anti-mouse antibodies (BD Pharmingen, San Diego, Calif. or eBioscience Inc., San Diego, Calif.). Analysis was performed on a FACSCalibur instrument using CellQuest software (version 4.0.2, BD, Franklin Lakes, N.J.).

Statistics. Data are expressed as mean±SEM for each group. Statistical differences between groups were evaluated by unpaired, one-tailed Student's t test using GraphPad Prism software (version 5.0a, GraphPad Software Inc., La Jolla, Calif.).

Results. After ischemia and reperfusion injury induction, type I NKT cells show increased IFN-γ production compared to type I NKT cells from sham surgery (FIG. 17). Administration of sulfatide 3 hrs prior to ischemia and reperfusion injury significantly reduced IFN-γ secretion by type I NKT cells (p<0.01) (FIG. 17).

### Example 16

### Evaluation of Lymphocyte Population in the Immune Response to Hepatic Ischemia and Reperfusion Injury

Changes in the proportion and absolute number of hepatic NK (NK1.1⁺TCRβ⁻) cells during ischemia and reperfusion injury were determined in untreated WT mice (WT), Jα18^{-/-} mice (Jα18^{-/-}), and sulfatide-treated WT mice (Sulfatide).

WT mice and Jα18^{-/-} mice (3-5 mice/group) or WT mice (2-3 mice/group) treated with 20 μg sulfatide/mouse i.p. 3 hrs prior to ischemia induction or sham surgery were subjected to 90 min of ischemia and 6 hrs of reperfusion or sham surgeries as described in Example 10.

Cell preparation. Leukocytes were isolated from murine cephalad liver lobes, using mechanical crushing followed by Percoll gradient separation and RBC lysis as described in Halder R C, Aguilera C, Maricic I, et al., Type II NKT cell-mediated anergy induction in type I NKT prevents inflammatory liver disease. J Clin Invest 2007, 117:2302-12.

Flow cytometry. Leukocytes were suspended in FACS buffer (PBS containing 0.02% NaN₃ and 2% FCS), blocked (anti-mouse FcR-γ, BD Pharmingen, San Diego, Calif.) and stained with loaded mCD1d-tetramer-PE or PE-, FITC-, or PE-Cy5-labeled anti-mouse antibodies (BD Pharmingen, San Diego, Calif. or eBioscience Inc., San Diego, Calif.) as indicated. Intracellular cytokine staining (ICCS) of liver mononuclear cells (MNCs) was carried out as described in Halder R C, Aguilera C, Maricic I, et al., Type II NKT cell-mediated anergy induction in type I NKT prevents inflammatory liver disease. J Clin Invest 2007, 117:2302-12. Analysis was performed on a FACSCalibur instrument using CellQuest software (version 4.0.2, BD, Franklin Lakes, N.J.).

Statistics. Data are expressed as mean±SEM for each group. Statistical differences between groups were evaluated by unpaired, one-tailed Student's t test using GraphPad Prism software (version 5.0a, GraphPad Software Inc., La Jolla, Calif.).

Results. Following staining of MNCs from cephalad liver lobes with mCD1d-tetramers or antibodies, the proportions and absolute numbers of NKT (NK1.1⁺TCRβ⁺) cells, type I NKT (αGalCer/tetramer⁺TCRβ⁺) cells, type II NKT (sulfatide/tetramer⁺TCRβ⁺) cells, CD4⁺ T cells, CD8⁺ T cells, and CD4⁻CD8⁻ T cells were determined (FIG. 18). At this reperfusion time point hepatic NKT cell subsets, CD8⁺ T cells and CD4⁻CD8⁻ T cells showed no significant changes in ischemia and reperfusion injury-induced WT mice compared to sham controls (FIG. 18). A slight reduction in type I NKT and total NKT cells was observed. CD4⁺ T cells were slightly, but significantly, decreased in livers from untreated WT (WT) and sulfatide-treated (Sulfatide) mice following 6 hrs of reperfusion.

As shown in FIG. 18 proportion of NK cells was significantly (p<0.01) increased in livers of WT^{mice} following 6 hrs of reperfusion compared to sham controls. In mice lacking type I NKT cells (Jα18^{-/-}); however, no significant change in NK cells was observed (FIG. 18), indicating that the increase in hepatic NK cells is dependent on the presence of type I NKT cells. Furthermore, in vivo activation of type II NKT cells by sulfatide prior to ischemia and reperfusion injury induction reduced the increase in hepatic NK cells (FIG. 18).

Changes in the proportion and absolute number of CD11b⁺Gr-1⁺ cells, including neutrophil granulocytes, and CD11b⁺Gr-1⁻ cells among liver leukocytes were analyzed in untreated WT mice, Jα18^{-/-} mice, and sulfatide-treated WT mice following 6 hrs of reperfusion or sham surgeries by flow cytometry (see FIG. 18). A significant increase in hepatic CD11b⁺ cells was observed in ischemia and reperfusion injury-induced WT mice compared to sham controls (37.8±3.9% vs. 23.4±1.8%, p<0.01). This increase included Gr-1⁺ cells comprising granulocytes, monocytes and myeloid precursor cells, as well as Gr-1⁻ cells mainly consisting of macrophages and myeloid dendritic cells but also of NK cells (FIG. 18).

### Example 17

### Analysis of the Effect of the Immune Response to Hepatic Ischemia and Reperfusion Injury on the Composition of the CD11b+Gr-1+ Cell Population

The CD11b⁺Gr-1⁺ population of cells can be further categorized into Gr-1^{int} cells, which are predominantly monocytes and myeloid precursors, and Gr-1^{high} cells, which are mainly granulocytes. To investigate whether the ischemia and reperfusion injury induced increase in the CD11b⁺Gr-1⁺ population is attributable to a particular subpopulation, the Gr-1^{high} and Gr-1^{int} subsets were analyzed separately by flow cytometry.

WT mice and Jα18^{-/-} mice (3-5 mice/group) or WT mice (2-3 mice/group) treated with 20 μg sulfatide/mouse i.p. 3 hrs prior to ischemia induction or sham surgery were subjected to 90 min of ischemia and 6 hrs of reperfusion or sham surgeries as described in Example 10.

Cell preparation. Leukocytes were isolated from the cephalad liver lobes of the mice using mechanical crushing followed by Percoll gradient separation and RBC lysis as described in Halder R C, Aguilera C, Maricic I, et al., Type II NKT cell-mediated anergy induction in type I NKT prevents inflammatory liver disease. J Clin Invest 2007, 117:2302-12.

Flow cytometry. Leukocytes were suspended in FACS buffer (PBS containing 0.02% NaN₃ and 2% FCS), blocked (anti-mouse FcR-γ, BD Pharmingen, San Diego, Calif.) and stained with loaded mCD1d-tetramer-PE or PE-, FITC-, or PE-Cy5-labeled anti-mouse antibodies (BD Pharmingen, San Diego, Calif. or eBioscience Inc., San Diego, Calif.) as indicated. Intracellular cytokine staining (ICCS) of liver mononuclear cells (MNCs) was carried out as described in Halder R C, Aguilera C, Maricic I, et al., Type II NKT cell-mediated anergy induction in type I NKT prevents inflammatory liver disease. J Clin Invest 2007, 117:2302-12. Analysis was performed on a FACSCalibur instrument using CellQuest software (version 4.0.2, BD, Franklin Lakes, N.J.). Gr-1^{high} and Gr-1^{int} populations were gated.

Statistics. Data are expressed as mean±SEM for each group. Statistical differences between groups were evaluated by unpaired, one-tailed Student's t test using GraphPad Prism software (version 5.0a, GraphPad Software Inc., La Jolla, Calif.).

Results. In the livers of WT mice following ischemia and reperfusion injury, the Gr-1^{int} subset of cells, predominantly comprising monocytes and myeloid precursors, is increased around 3.5-fold (p<0.005), while the Gr-1^{high} subset, mainly consisting of granulocytes, did not differ significantly between livers of sham controls and ischemia and reperfusion injury-induced mice (FIG. 19).

The increase in the Gr-1^{int} subset of cells following ischemia and reperfusion injury was not observed in Jα18^{-/-} mice (FIG. 18 and FIG. 19). Therefore, the hepatic recruitment of myeloid cell subsets during ischemia and reperfusion injury is dependent on the presence of type I NKT cells, which are lacking in Jα18^{-/-} mice. The in vivo activation of type II NKT cells by sulfatide 3 hrs prior to ischemia and reperfusion injury induction diminished hepatic recruitment of myeloid cells, as proportions of CD11b⁺Gr-1⁺ and CD11b⁺Gr-1⁻ populations were not significantly altered between sulfatide-treated sham and ischemia and reperfusion injury mice, while CD11b⁺Gr-1^{int} cells were reduced by ˜50% in sulfatide-treated mice compared to untreated mice (p<0.05) (FIG. 18 and FIG. 19).

### Example 18

### Levels of Serum ALT Induced in Response to Ischemia and Reperfusion Injury are Reduced Following Administration of Cis-Tetracosenol Sulfatide

Alanine aminotransferase (ALT) enzyme levels, a marker of heptocelluar damage, were assessed in mice treated with cis-tetracosenoyl sulfatide or PBS and subjected to ischemia and reperfusion injury.

Cis-tetracosenoyl sulfatide was dissolved in vehicle (0.5% polysorbate-20 (Tween-20) and 0.9% NaCl solution) and diluted in PBS. Groups (2 mice per group) of BL/6 mice were treated with either cis-tetracosenoyl sulfatide (20 μg/mouse) or PBS by a single intraperitoneal injection 3 hrs prior to ischemia induction. Hepatic ischemia and reperfusion injury was induced as described in Example 10. Following 6 hrs of reperfusion, blood was obtained by cardiac puncture and serum was isolated. Serum Alanine Aminotransferase (ALT) levels were determined on Olympus 5400 chemistry analyzer (IDEXX Laboratories Inc., Westbrook, Me.).

Average ALT levels of 5901+0 U/I were observed in mice treated with PBS prior to hepatic ischemia and reperfusion injury, while in the group of mice treated with cis-tetracosenoyl sulfatide prior to hepatic ischemia and reperfusion injury, average ALT levels were 2499+0 U/I. This represents a more than a 55% reduction in ALT levels in mice treated with a single injection of cis-tetracosenoyl sulfatide prior to hepatic ischemia and reperfusion injury compared to the control.

### EQUIVALENTS

The foregoing written specification is considered to be sufficient to enable one skilled in the art to practice the present embodiments. The foregoing description details certain preferred embodiments and describes the best mode contemplated by the inventors. It will be appreciated, however, that no matter how detailed the foregoing may appear in text, the present embodiments may be practiced in many ways and should be construed in accordance with the appended claims and any equivalents thereof.

What is claimed is:

1. A method for treating or preventing at least one of hepatic ischemia and hepatic reperfusion injury associated with a surgical procedure in a patient comprising administering an effective amount of sulfatide to the patient prior to the surgical procedure.

2. The method of claim 1, wherein the sulfatide is administered from about 1 hour to about 24 hours prior to the surgical procedure.

3. The method of claim 1, wherein the sulfatide is administered from about 2 hours to about 8 hours prior to the surgical procedure.

4. The method of claim 1, wherein the sulfatide is administered about 6 hours prior to the surgical procedure.

5. The method of claim 1, wherein the amount of sulfatide administered is about 1μ gram/kg of body weight.

6. The method of claim 1, wherein the sulfatide has following chemical structure: 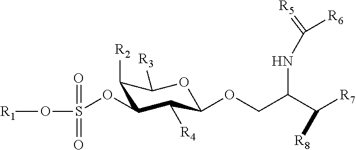
wherein R₁ is selected from the group consisting of a bond, a hydrogen, a C₁ to C₃₀ alkyl, C₁ to C₃₀ substituted alkyl, a C₁ to C₃₀ alkenyl, a C₁ to C₃₀ substituted alkenyl and a C₅ to C₁₂ sugar; R₂ is selected from the group consisting of a hydrogen, a hydroxy group, a methoxy group, and an alkoxy group; R₃ is selected from the group consisting of a hydrogen, a hydroxy group, a methoxy group, an ethoxy group, and an alkoxy group; R₄ is selected from the group consisting of a hydrogen, a hydroxy group and an alkoxy group; R₅ is selected from the group consisting of a hydrogen, a hydroxyl, a carbonyl, an alkoxy and a bond; R₆ is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; R₇ is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; and R₈ is selected from the group consisting of a hydrogen, a hydroxyl group, a carbonyl, an alkoxy group and a bond.

7. The method of claim 1, wherein the sulfatide has following chemical structure: 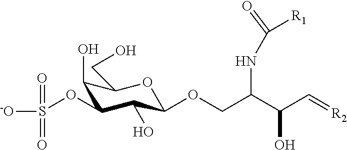
wherein R₁ is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; and R₂ is selected from the group consisting of a hydrogen, a hydroxyl group, a carbonyl, an alkoxy group and a bond.

8. The method of claim 1, wherein the sulfatide has the following chemical structure:

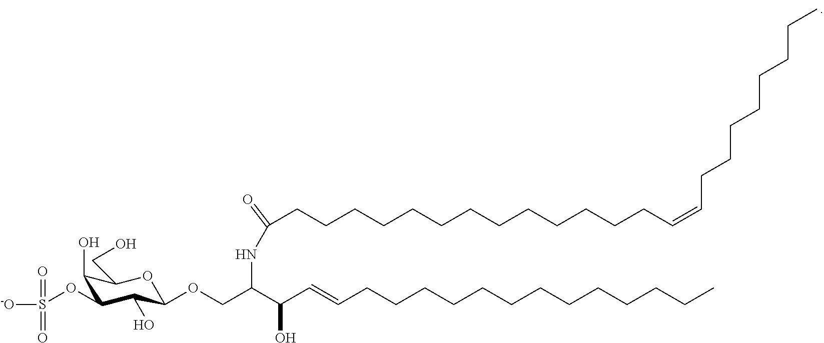

9. The method of claim 1, further comprising administering sulfatide during the surgical procedure and/or after the surgical procedure.

10. A method for preventing hepatic ischemia and reperfusion injury comprising administering an agent which inhibits the activity of type I NKT by activating type II NKT.

11. The method of claim 10, wherein type II NKT cells are activated by administering a sulfatide.

12. The method of claim 11, wherein the sulfatide is administered 6 hours prior to a surgical procedure.

13. The method of claim 11, wherein the sulfatide has following chemical structure: 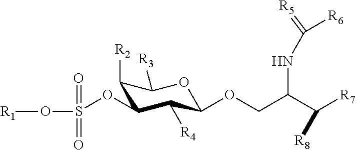
wherein R1 is selected from the group consisting of a bond, a hydrogen, a C₁ to C₃₀ alkyl, C₁ to C₃₀ substituted alkyl, a C₁ to C₃₀ alkenyl, a C₁ to C₃₀ substituted alkenyl and a C₅ to C₁₂ sugar; R₂ is selected from the group consisting of a hydrogen, a hydroxy group, a methoxy group, and an alkoxy group; R₃ is selected from the group consisting of a hydrogen, a hydroxy group, a methoxy group, an ethoxy group, and an alkoxy group; R₄ is selected from the group consisting of a hydrogen, a hydroxy group and an alkoxy group; R₅ is selected from the group consisting of a hydrogen, a hydroxyl, a carbonyl, an alkoxy and a bond; R₆ is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; R₇ is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; and R₈ is selected from the group consisting of a hydrogen, a hydroxyl group, a carbonyl, an alkoxy group and a bond.

14. The method of claim 11, wherein the sulfatide has following chemical structure: 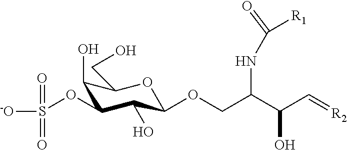
wherein R₁ is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; and R₂ is selected from the group consisting of a hydrogen, a hydroxyl group, a carbonyl, an alkoxy group and a bond.

15. The method of claim 11, wherein the sulfatide has the following chemical structure:

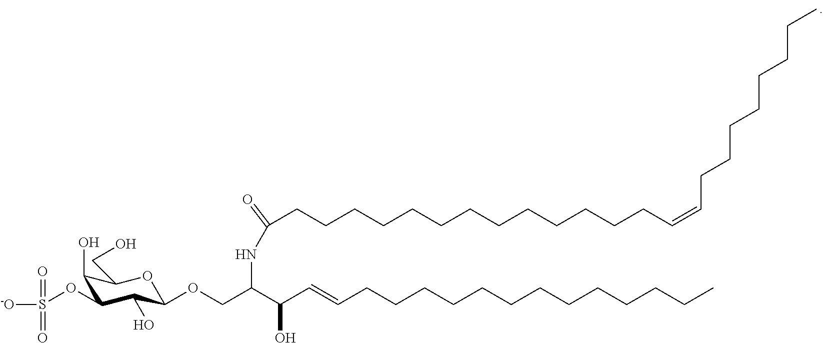

16. A method for evaluating the effectiveness of a sulfatide in preventing ischemia and reperfusion injury comprising:
a) administering an effective amount of the sulfatide to a subject;
b) inducing a hepatic ischemia/reperfusion injury; and
c) assessing a liver sample taken from the subject after 6 hours of reperfusion for markers of ischemia and reperfusion injury.

17. The method of claim 16, wherein the marker of reperfusion injury comprises elevated levels of Alanine aminotransferase (ALT) enzyme.

18. The method of claim 16, wherein the marker of reperfusion injury comprises IFN-γ secretion.

19. The method of claim 16, wherein the sulfatide has following chemical structure: 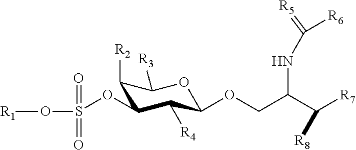
wherein R1 is selected from the group consisting of a bond, a hydrogen, a C₁ to C₃₀ alkyl, C₁ to C₃₀ substituted alkyl, a C₁ to C₃₀ alkenyl, a C₁ to C₃₀ substituted alkenyl and a C₅ to C₁₂ sugar; R₂ is selected from the group consisting of a hydrogen, a hydroxy group, a methoxy group, and an alkoxy group; R₃ is selected from the group consisting of a hydrogen, a hydroxy group, a methoxy group, an ethoxy group, and an alkoxy group; R₄ is selected from the group consisting of a hydrogen, a hydroxy group and an alkoxy group; R₅ is selected from the group consisting of a hydrogen, a hydroxyl, a carbonyl, an alkoxy and a bond; R₆ is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; R₇ is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; and R₈ is selected from the group consisting of a hydrogen, a hydroxyl group, a carbonyl, an alkoxy group and a bond.

20. A method of treating or preventing ischemia or reperfusion injury in a patient comprising administering an effective amount of sulfatide to the patient in need thereof.

21. The method of claim 20, wherein the patient is a surgical patient.

22. The method of claim 20, wherein the ischemia or reperfusion injury is associated with at least one of myocardial infarction, arteriosclerosis, stroke, septic shock, traumatic shock, vascular interventional procedures, angioplasty, abdominal surgery, abdominoplasty, adenoidectomy, amputation, appendectomy, arthrodesis, arthroplasty, sickle cell anemia, brain surgery, cesarean section, cholecystectomy, colon resection, colostomy, corneal transplantation, discectomy, endarterectomy, gastrectomy, grafting of skin or other tissues, heart transplantation, heart surgery, hemicorporectomy, hemorrhoidectomy, hepatectomy, hernia repair, hysterectomy, kidney transplantation, laminectomy, laryngectomy, crush injury, myocardial infarction, stroke, brain trauma, hepatic surgery, hepatic injury, renal surgery, renal injury, lumpectomy, liver transplantation, lung transplantation, kidney transplantation, mammoplasty, mastectomy, mastoidectomy, myotomy, nephrectomy, nissen fundoplication, oophorectomy, orchidectomy, orthopedic surgery, parathyroidectomy, plastic surgery, penectomy, phalloplasty, pneumonectomy, prostatectomy, radiosurgery, rotationplasty, splenectomy, stapedectomy, thoracotomy, thrombectomy, thymectomy, thyroidectomy, tonsillectomy, ulnar collateral ligament reconstruction, vaginectomy, vasectomy, cardiac bypass, cardiac artery bypass, graft surgery and organ transplantation.

23. The method of claim 20, wherein the ischemia or reperfusion injury is related to liver transplantation or liver injury.

24. The method of claim 20, wherein the sulfatide is administered intraperitoneally.

25. The method of claim 20, wherein the sulfatide is administered intravenously.

26. The method of claim 20, wherein the sulfatide is administered prior to a surgical procedure.

27. The method of claim 20, wherein the sulfatide is administered during or after a surgical procedure.

28. The method of claim 20, wherein the sulfatide has following chemical structure: 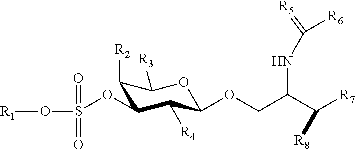
wherein R₁ is selected from the group consisting of a bond, a hydrogen, a C₁ to C₃₀ alkyl, C₁ to C₃₀ substituted alkyl, a C₁ to C₃₀ alkenyl, a C₁ to C₃₀ substituted alkenyl and a C₅ to C₁₂ sugar; R₂ is selected from the group consisting of a hydrogen, a hydroxy group, a methoxy group, and an alkoxy group; R₃ is selected from the group consisting of a hydrogen, a hydroxy group, a methoxy group, an ethoxy group, and an alkoxy group; R₄ is selected from the group consisting of a hydrogen, a hydroxy group and an alkoxy group; R₅ is selected from the group consisting of a hydrogen, a hydroxyl, a carbonyl, an alkoxy and a bond; R₆ is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; R₇ is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; and R₈ is selected from the group consisting of a hydrogen, a hydroxyl group, a carbonyl, an alkoxy group and a bond.

29. The method of claim 20, wherein the sulfatide has following chemical structure: 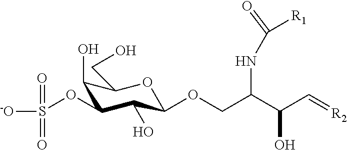
wherein R₁ is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; and R₂ is selected from the group consisting of a hydrogen, a hydroxyl group, a carbonyl, an alkoxy group and a bond.

30. The method of claim 20, wherein the sulfatide has the following chemical structure: [chemical structure]
